(19) United States
(12) Patent Application Publication
(10) Pub. No.: US 2013/0289014 A1
(43) Pub. Date: Oct. 31, 2013

(54) Combination therapy in treatment of oncological and fibrotic diseases

(71) Applicants: Flavio SOLCA, Vienna (AT); Ulrich GUERTLER, Oepfingen (DE); Ulrike TONTSCH-GRUNT, Baden (AT)

(72) Inventors: Flavio SOLCA, Vienna (AT); Ulrich GUERTLER, Oepfingen (DE); Ulrike TONTSCH-GRUNT, Baden (AT)

(73) Assignee: BOEHRINGER INGELHEIM INTERNATIONAL GMBH, Ingelheim am Rhein (DE)

(21) Appl. No.: 13/930,232

(22) Filed: Jun. 28, 2013

Related U.S. Application Data
(63) Continuation of application No. 13/089,696, filed on Apr. 19, 2011, now abandoned.

(30) Foreign Application Priority Data
Apr. 27, 2010 (EP) 10161229.9

(51) Int. Cl. A61K 31/496; A61K 31/506; A61K 45/06; A61K 31/5377

(52) U.S. Cl. CPC A61K 31/496; A61K 31/5377; A61K 31/506; A61K 45/06
USPC 514/210.18; 514/254.09; 514/252.14; 514/235.8; 514/252.18

(57) ABSTRACT

The invention relates to new methods for the treatment of oncological and fibrotic disease comprising the combined administration of a cell signalling and/or angiogenesis inhibitor in conjunction with an Aurora kinase inhibitor.

The invention relates to new methods for the treatment of oncological and fibrotic diseases comprising the combined administration of a cell signalling and/or angiogenesis inhibitor, particularly an inhibitor of vascular endothelial growth factor receptors (VEGFRs) in conjunction with an Aurora kinase inhibitor (AKI), as well as to pharmaceutical combinations or compositions comprising such active ingredients.

The compound (3-Z-[1-(4-(N-((4-methyl-piperazin-1-yl)-methylcarbonyl)-N-methyl-amino)-anilino)-1-phenyl-methylene]-6-methoxycarbonyl-2-indolinone), hereinafter referred to as BIBF 1120, is an innovative active ingredient having valuable pharmacological properties, especially for the treatment of oncological and fibrotic diseases, immunologic diseases or pathological conditions involving an immunologic component, or fibrotic diseases. The chemical structure of this compound is depicted below as formula 1

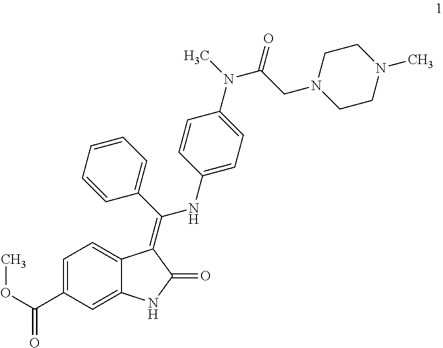 [SMILES: COC(=O)c1ccc2c(c1)NC(=O)/C2=C(\Nc1ccc(N(C)C(=O)CN2CCN(C)CC2)cc1)c1ccccc1]

The base form of this compound is described in WO 01/27081, the monoethanesulphonate salt form is described in WO 2004/013099 and various further salt forms are presented in WO 2007/141283. The use of this molecule for the treatment of immunologic diseases or pathological conditions involving an immunologic component is being described in WO 2004/017948, the use for the treatment of oncological diseases is being described in WO 2004/096224 and the use for the treatment of fibrotic diseases is being described in WO 2006/067165.

BIBF 1120 is a highly potent, orally bioavailable triple angiokinase inhibitor that inhibits three growth factor receptors simultaneously: vascular endothelial growth factor receptor (VEGFR), platelet-derived growth factor receptor (PDGFR) and fibroblast growth factor receptor (FGFR). All three growth factors are crucially involved in the formation of blood vessels (angiogenesis) and inhibition of them may play a critical role in the prevention, inhibition or suppression of tumour neovascularization, tumour growth and spread (metastases). BIBF 1120's inhibition of VEGFR and FGFR is thought to have an impact on the formation of new tumour blood vessels and its inhibition of FGFR and PDGFR may have an effect on the maintenance of the tumour vascular integrity. It has been shown that this compound suppresses tumor growth through mechanisms inhibiting tumor neovascularization and inhibits signalling in endothelial- and smooth muscle cells and pericytes, and reduces tumor vessel density. BIBF 1120 is thus suitable for the treatment of diseases in which angiogenesis or the proliferation of cells is involved.

The serine/threonine kinase Aurora B is involved in the regulation of several mitotic processes, including chromosome condensation, congression and segregation as well as cytokinesis. Inactivation of Aurora B abrogates the spindle assembly checkpoint (SAC) and causes premature mitotic exit without cytokinesis, resulting in polyploid cells that eventually stop further DNA replication. Aurora B inhibitors induce a mitotic override (mitotic slippage). Compound X, a potent inhibitor of Aurora B kinase according to this invention, blocks proliferation in various human cancer cell lines and induces polyploidy, senescence and apoptosis. Compound X shows excellent in vivo activity in multiple cancer xenograft models in nude mice.

The purpose of the instant invention is the provision of a new therapy for the treatment of oncological and fibrotic diseases.

### DETAILED DESCRIPTION OF THE INVENTION

The invention relates to new methods for the treatment of oncological and fibrotic diseases comprising the combined administration of a cell signalling and/or angiogenesis inhibitor, particularly a compound of formula 1 (BIBF 1120)

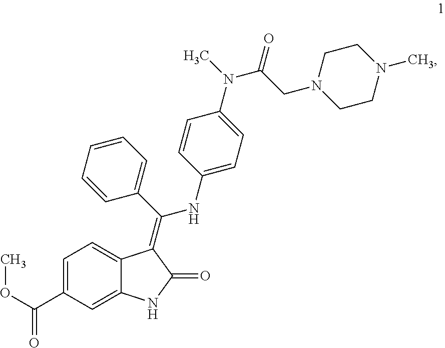 [SMILES: COC(=O)c1ccc2c(c1)NC(=O)/C2=C(\Nc1ccc(N(C)C(=O)CN2CCN(C)CC2)cc1)c1ccccc1]

optionally in the form of the tautomers and pharmaceutically acceptable salts thereof, and an Aurora kinase inhibitor, particularly an inhibitor of Aurora B kinase.

Within this invention it is to be understood that the combinations, compositions or combined uses according to this invention may envisage the simultaneous, sequential or separate administration of the active ingredients. It will be appreciated that the cell signalling and/or angiogenesis inhibitor and the Aurora kinase inhibitor can be administered formulated either dependently or independently, such as e.g. the cell signalling and/or angiogenesis inhibitor and the Aurora kinase inhibitor may be administered either as part of the same pharmaceutical composition/dosage form or in separate pharmaceutical compositions/dosage forms.

In this context, “combination” or “combined” within the meaning of this invention includes, without being limited, fixed and non-fixed (e.g. free) forms (including kits) and uses, such as e.g. the simultaneous, sequential or separate use of the components or ingredients.

The administration of the cell signalling and/or angiogenesis inhibitor and the Aurora kinase inhibitor may take place by administering the active components or ingredients together, such as e.g. by administering them simultaneously in one single or in two separate formulations or dosage forms. Alternatively, the administration of the cell signalling and/or angiogenesis inhibitor and the Aurora kinase inhibitor may take place by administering the active components or ingredients sequentially, such as e.g. successively in two separate formulations or dosage forms.

Cell signalling and/or angiogenesis inhibitors may include, without being limited, agents targeting (e g inhibiting) endothelial-specific receptor tyrosine kinase (Tie-2), epidermal growth factor receptor (EGFR), insulin-like growth factor-1 receptor (IGF-1R), fibroblast growth factor receptor (FGFR), platelet-derived growth factor receptor (PDGFR), or vascular endothelial growth factor (VEGF) or VEGF receptor (VEGFR); as well as thrombospondin analogs, matrix metalloprotease (e.g. MMP-2 or MMP-9) inhibitors, thalidomide or thalidomide analogs, integrins, angiostatin, endostatin, vascular disrupting agents (VDA), protein kinase C (PKC) inhibitors, and the like.

Particular angiogenesis inhibitors of this invention are agents targeting (e g inhibiting) vascular endothelial growth factor (VEGF) or VEGF receptor (VEGFR).

Agents targeting (e.g. inhibiting) VEGF/VEGFR relate to compounds which target (e g inhibit) one or more members of the VEGF or VEGFR family (VEGFR1, VEGFR2, VEGFR3) and include inhibitors of any vascular endothelial growth factor (VEGF) ligand (such as e.g. ligand antibodies or soluble receptors) as well as inhibitors of any VEGF receptor (VEGFR) (such as e.g. VEGFR tyrosin kinase inhibitors, VEGFR antagonists or receptor antibodies).

A VEGFR inhibitor is an agent that targets one or more members of the family of vascular endothelial growth factor (VEGF) receptor, particularly of the VEGFR family of tyrosine kinases (either as single kinase inhibitor or as multikinase inhibitor), including small molecule receptor tyrosine kinase inhibitors and anti-VEGFR antibodies.

Examples of small molecule VEGFR inhibitors include, without being limited to, sorafenib (Nexavar, also an inhibitor of Raf, PDGFR, Flt3, Kit and RETR), sunitinib (Sutent, also inhibitor of Kit, Flt3 and PDGFR), pazopanib (GW-786034, also inhibitor of Kit and PDGFR), cediranib (Recentin, AZD-2171), axitinib (AG-013736, also inhibitor of PDGFR and Kit), vandetanib (Zactima, ZD-6474, also inhibitor of EGFR and Ret), vatalanib (also inhibitor of PDGFR and Kit), motesanib (AMG-706, also inhibitor of PDGFR and Kit), brivanib (also FGFR inhibitor), linifanib (ABT-869, also inhibitor of PDGFR, Flt3 and Kit), tivozanib (KRN-951, also inhibitor of PDGFR, Kit, and MAP), E-7080 (also inhibitor of Kit and Kdr), regorafenib (BAY-73-4506, also inhibitor of Tek), foretinib (XL-880, also inhibitor of Flt3, Kit and Met), telatinib (BAY-57-9352), MGCD-265 (also inhibitor of c-MET, Tie2 and Ron), dovitinib (also inhibitor of PDGFR, Flt3, Kit and FGFR), BIBF-1120 (also inhibitor of FGFR and PDGFR), XL-184 (also inhibitor of Met, Flt3, Ret, Tek and Kit).

Examples of biological entities inhibiting VEGF(R) include, without being limited to, anti-VEGF ligand antibodies such as e.g. bevacizumab (Avastin); soluble receptors such as aflibercept (VEGF-Trap); anti-VEGF receptor antibodies such as e.g. ramucirumab (IMC-1121b) or IMC-18F1; VEGFR antagonists such as e.g. CT-322 or CDP-791.

Examples of small molecule VEGFR-1 (Flt-1) inhibitors include, without being limited to, sunitinib, cediranib and dovitinib.

Examples of small molecule VEGFR-2 (Flk-1, Kdr) inhibitors include, without being limited to, sorafenib, sunitinib, cediranib and dovitinib.

Examples of small molecule VEGFR-3 (Flt-4) inhibitors include, without being limited to, sorafenib, sunitinib and cediranib.

Agents targeting (e.g. inhibiting) PDGFR relate to compounds which target (e g inhibit) one or more members of the PDGFR family and include inhibitors of a platelet-derived growth factor receptor (PDGFR) family tyrosin kinase (either as single kinase inhibitor or as multikinase inhibitor) as well as anti-PDGFR antibodies.

A PDGFR inhibitor is an agent that targets one or more members of the PDGFR family, particularly of the PDGFR family of tyrosine kinases (either as single kinase inhibitor or as multikinase inhibitor), including small molecule receptor tyrosine kinase inhibitors and anti-PDGFR antibodies.

Examples of small molecule PDGFR inhibitors include, without being limited to, BIBF-1120 (also inhibitor of VEGFR and FGFR), axitinib (also inhibitor of VEGFR and Kit), dovitinib (also inhibitor of VEGFR, Flt3, Kit and FGFR), sunitinib (also inhibitor of VEGFR, Flt3 and Kit), motesanib (also inhibitor of VEGFR and Kit), pazopanib (also inhibitor of VEGFR and Kit), nilotinib (also inhibitor of Abl and Kit), tandutinib (also inhibitor of Flt3 and Kit), vatalanib (also inhibitor of VEGFR and Kit), tivozanib (KRN-951, also inhibitor of VEGFR, Kit, and MAP), AC-220 (also inhibitor of Flt3 and Kit), TSU-68 (also inhibitor of FGFR and VEGFR), KRN-633 (also inhibitor of VEGFR, Kit and Flt3), linifinib (also inhibitor of Flt3, Kit and VEGFR), sorafenib (Nexavar, also an inhibitor of Raf, VEGFR, Flt3, Kit and RETR), imatinib (Glevec, also inhibitor of Abl and Kit). Examples of anti-PDGFR antibodies include, without being limited to, IMC-3G3.

Agents targeting FGFR relate to compounds which target one or more members of the FGFR family and include inhibitors of a fibroblast growth factor receptor family tyrosin kinase (either as single kinase inhibitor or as multikinase inhibitor).

A FGFR inhibitor is an agent that targets one or more members of the FGFR family (e.g. FGFR1, FGFR2, FGFR3), particularly of the FGFR family of tyrosine kinases (either as single kinase inhibitor or as multikinase inhibitor), including small molecule receptor tyrosine kinase inhibitors and anti-FGFR antibodies.

Examples of small molecule FGFR inhibitors include, without being limited to, BIBF-1120 (also inhibitor of VEGFR and PDGFR), dovitinib (also inhibitor of VEGFR, Flt3, Kit and PDGFR), KW-2449 (also inhibitor of Flt3 and Abl), brivanib (also VEGFR inhibitor), TSU-68 (also inhibitor of PDGFR and VEGFR).

Agents targeting (e.g. inhibiting) EGFR relate to compounds which target (e g inhibit) one or more members of the epidermal growth factor receptor family (erbB 1, erbB2, erbB3, erbB4) and include inhibitors of one or more members of the epidermal growth factor receptor (EGFR) family kinases (either as single kinase inhibitor or as multikinase inhibitor) as well as antibodies binding to one or more members of the epidermal growth factor receptor (EGFR) family.

A EGFR inhibitor is an agent that targets one or more members of the EGFR family, particularly of the EGFR family of tyrosine kinases (either as single kinase inhibitor or as multikinase inhibitor), including small molecule receptor tyrosine kinase inhibitors and anti-EGFR antibodies.

Examples of small molecule epidermal growth factor receptor (EGFR) inhibitors include, without being limited to, erlotinib (Tarceva), gefitinib (Iressa), BIBW-2992, lapatinib (Tykerb), vandetanib (Zactima, also inhibitor of VEGFR and RETR), neratinib (HKI-272), varlitinib, AZD-8931, AC-480, AEE-788 (also inhibitor of VEGFR).

Examples of antibodies against the epidermal growth factor receptor (EGFR) include, without being limited to, the anti-ErbB 1 antibodies cetuximab, panitumumab or nimotuzumab, the anti-ErbB 2 antibodies trastuzumab (Herceptin), pertuzumab (Omnitarg) or ertumaxomab, and the anti-EGFR antibody zalutumumab.

EGFR inhibitors in the meaning of this invention may refer to reversible EGFR tyrosin kinase inhibitors, such as e.g. gefitinib, erlotinib, vandetanib or lapatinib, or to irreversible EGFR tyrosin kinase inhibitors, such as e.g. neratinib or PF-299804.

EGFR inhibitors in the meaning of this invention may refer to erbB selective inhibitors, such as e.g. erbB1 inhibitors (e.g. erlotinib, gefitinib, cetuximab, panitumumab), or erbB2 inhibitors (e.g. trastuzumab), dual erbB 1/erbB2 inhibitors (e.g. lapatinib, BIBW-2992) or pan-erbB inhibitors (e.g. PF-299804).

IGF(R) inhibitors are agents that target one or more members of the insulin-like growth factor (IGF) family, particularly of the IGFR family of tyrosine kinases, e.g. IGFR-1 (either as single kinase inhibitor or as multikinase inhibitor), and/or insulin receptor pathways, and may include, without being limited to, the IGFR tyrosin kinase inhibitors BMS-754807 and OSI-906, as well as the an anti-IGF(R) antibodies figitumumab, cixutumumab, dalotuzumab and robatumumab.

Vascular targeting agents (VTAs) may include, without being limited to, vascular damaging or disrupting agents such as e.g. 5,6-dimethylxanthenone-4-acetic acid (DMXAA, vadimezan), combretastatin A4 phosphate (Zybrestat) or combretastatin A4 analogues, such as e.g. ombrabulin (AVE-8062).

Thrombospondin analogs may include, without being limited to, ABT-510.

Matrix metalloprotease (MMP) inhibitors may include, without being limited to, marimastat.

PKC inhibitors are agents that inhibit one or more members of the protein kinase C (PKC) family (either as single kinase inhibitor or as multikinase inhibitor) and may include, without being limited to, enzastaurin, bryostatin and midostaurin.

In an embodiment, a cell signalling and/or angiogenesis inhibitor of this invention refers preferably to an angiogenesis inhibitor, such as e.g. an agent targeting VEGF or VEGFR.

Preferred angiogenesis inhibitors of this invention may be selected from bevacizumab (Avastin), aflibercept (VEGF-Trap), vandetanib, cediranib, axitinib, sorafenib, sunitinib, motesanib, vatalanib, pazopanib, dovitinib and BIBF 1120.

A more preferred angiogenesis inhibitor of this invention is BIBF 1120.

In a further embodiment, a cell signalling and/or angiogenesis inhibitor of this invention refers preferably to a cell signalling inhibitor, such as e.g. an agent targeting EGFR.

A preferred cell signalling inhibitor of this invention is BIBW-2992.

In one embodiment (embodiment A), examples of Aurora kinase inhibitors may be found in WO 2007/003596, WO 2007/122219, WO 2007/132010, WO 2008/077885, WO 2008/152013, WO 2008/152014 and WO 2010/012747, the disclosures of which are incorporated herein by reference in their entireties.

In a particular sub-embodiment of embodiment A, the Aurora kinase inhibitor is selected from the group consisting of the compounds (pyrimidine or indolinone derivatives) of the following Table i (compounds 1 to 36), optionally in the form of the tautomers and pharmaceutically acceptable salts thereof.

TABLE i
AKI Compounds No. 1-36:

| No. | AKI compound |
|---|---|
| 1 | 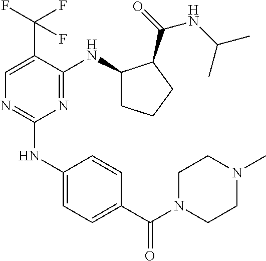 [SMILES: CC(C)NC(=O)[C@H]1CCC[C@H]1Nc1nc(Nc2ccc(C(=O)N3CCN(C)CC3)cc2)ncc1C(F)(F)F] |
| 2 | 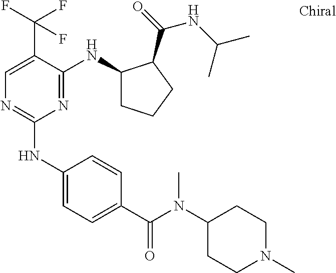 [SMILES: CC(C)NC(=O)[C@H]1CCC[C@H]1Nc1nc(Nc2ccc(C(=O)N(C)C3CCN(C)CC3)cc2)ncc1C(F)(F)F] |
| 3 | 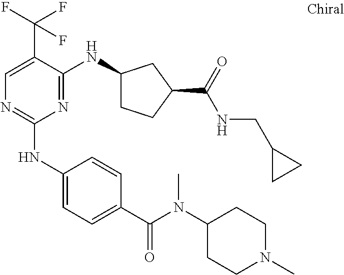 [SMILES: CN1CCC(N(C)C(=O)c2ccc(Nc3ncc(C(F)(F)F)c(N[C@@H]4CC[C@H](C(=O)NCC5CC5)C4)n3)cc2)CC1] |
| 4 | 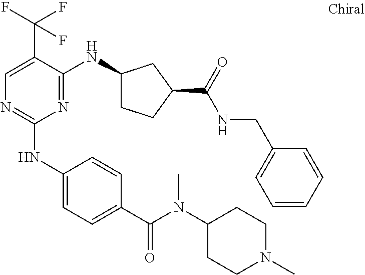 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1] |
| 5 | 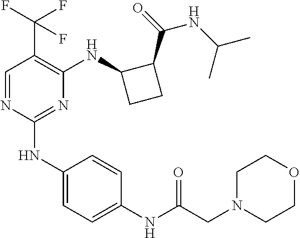 [SMILES: CC(C)NC(=O)[C@H]1CC[C@H]1Nc1nc(Nc2ccc(NC(=O)CN3CCOCC3)cc2)ncc1C(F)(F)F] |
| 6 | 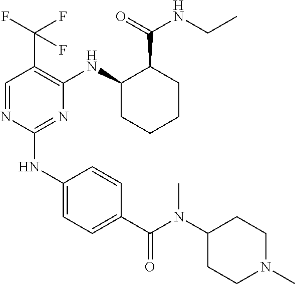 [SMILES: CCNC(=O)[C@H]1CCCC[C@H]1Nc1nc(Nc2ccc(C(=O)N(C)C3CCN(C)CC3)cc2)ncc1C(F)(F)F] |
| 7 | 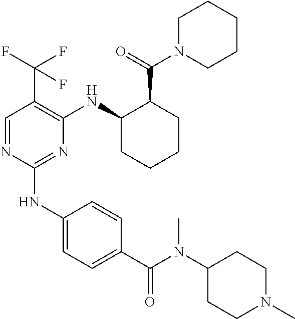 [SMILES: CN1CCC(N(C)C(=O)c2ccc(Nc3ncc(C(F)(F)F)c(N[C@@H]4CCCC[C@@H]4C(=O)N4CCCCC4)n3)cc2)CC1] |
| 8 | 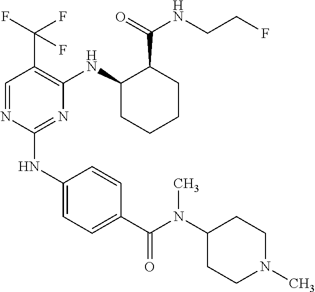 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 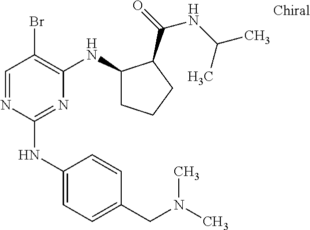 [SMILES: CC(C)NC(=O)[C@H]1CCC[C@H]1Nc1nc(Nc2ccc(CN(C)C)cc2)ncc1Br] |
| 10 | 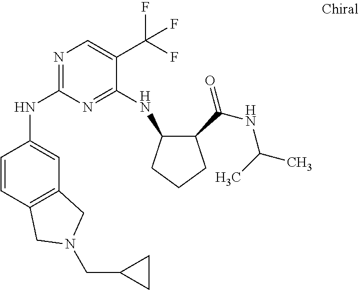 [SMILES: CC(C)NC(=O)[C@H]1CCC[C@H]1Nc1nc(Nc2ccc3c(c2)CN(CC2CC2)C3)ncc1C(F)(F)F] |
| 11 | 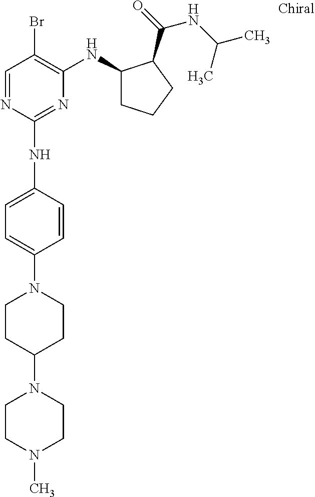 [SMILES: CC(C)NC(=O)[C@H]1CCC[C@H]1Nc1nc(Nc2ccc(N3CCC(N4CCN(C)CC4)CC3)cc2)ncc1Br] |
| 12 | 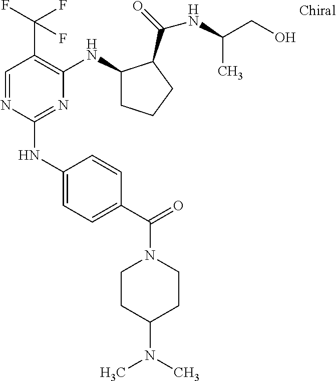 [SMILES: C[C@H](CO)NC(=O)[C@H]1CCC[C@H]1Nc1nc(Nc2ccc(C(=O)N3CCC(N(C)C)CC3)cc2)ncc1C(F)(F)F] |
| 13 | 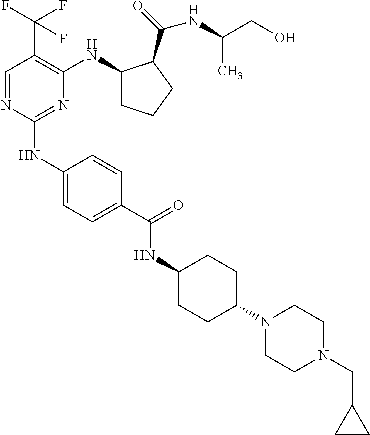 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F] |
| 14 | 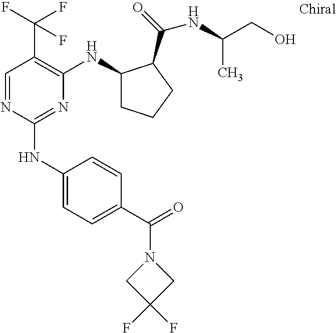 [SMILES: C[C@H](CO)NC(=O)[C@H]1CCC[C@H]1Nc1nc(Nc2ccc(C(=O)N3CC(F)(F)C3)cc2)ncc1C(F)(F)F] |
| 15 | 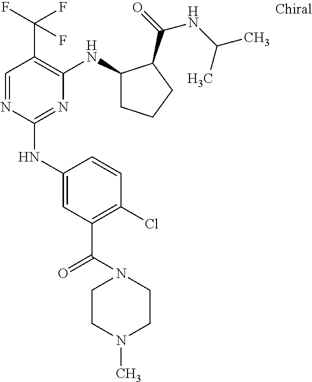 [SMILES: CC(C)NC(=O)[C@H]1CCC[C@H]1Nc1nc(Nc2ccc(Cl)c(C(=O)N3CCN(C)CC3)c2)ncc1C(F)(F)F] |
| 16 | 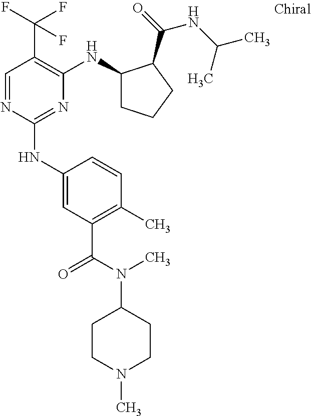 [SMILES: Cc1ccc(Nc2ncc(C(F)(F)F)c(N[C@@H]3CCC[C@@H]3C(=O)NC(C)C)n2)cc1C(=O)N(C)C1CCN(C)CC1] |
| 17 | 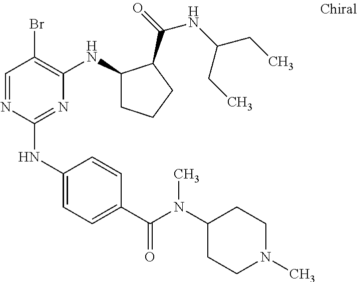 [SMILES: CCC(CC)NC(=O)[C@H]1CCC[C@H]1Nc1nc(Nc2ccc(C(=O)N(C)C3CCN(C)CC3)cc2)ncc1Br] |
| 18 | 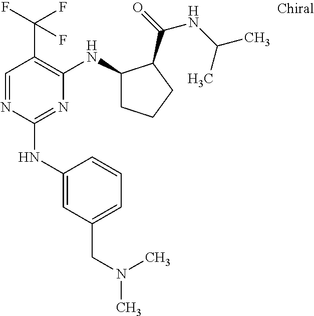 [SMILES: CC(C)NC(=O)[C@H]1CCC[C@H]1Nc1nc(Nc2cccc(CN(C)C)c2)ncc1C(F)(F)F] |
| 19 | 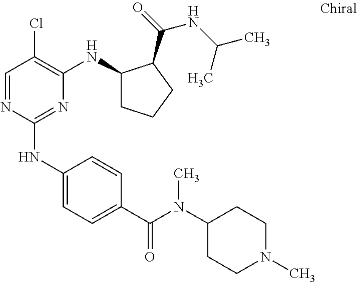 [SMILES: CC(C)NC(=O)[C@H]1CCC[C@H]1Nc1nc(Nc2ccc(C(=O)N(C)C3CCN(C)CC3)cc2)ncc1Cl] |
| 20 | 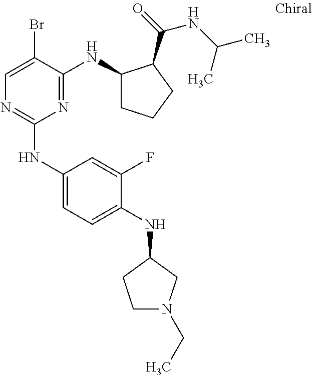 [SMILES: CCN1CC[C@@H](Nc2ccc(Nc3ncc(Br)c(N[C@@H]4CCC[C@@H]4C(=O)NC(C)C)n3)cc2F)C1] |
| 21 | 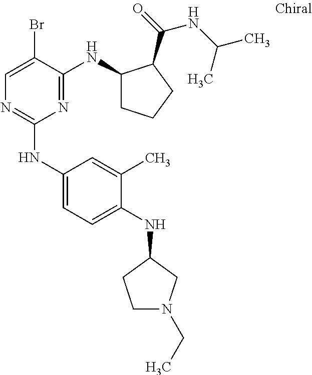 [SMILES: CCN1CC[C@@H](Nc2ccc(Nc3ncc(Br)c(N[C@@H]4CCC[C@@H]4C(=O)NC(C)C)n3)cc2C)C1] |
| 22 | 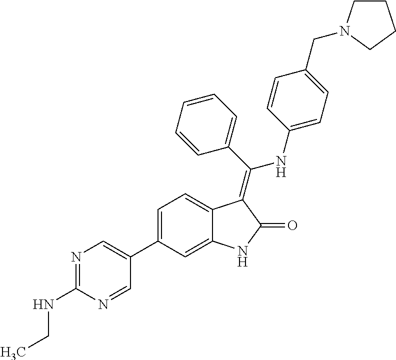 [SMILES: CCNc1ncc(-c2ccc3c(c2)NC(=O)/C3=C(\Nc2ccc(CN3CCCC3)cc2)c2ccccc2)cn1] |
| 23 | 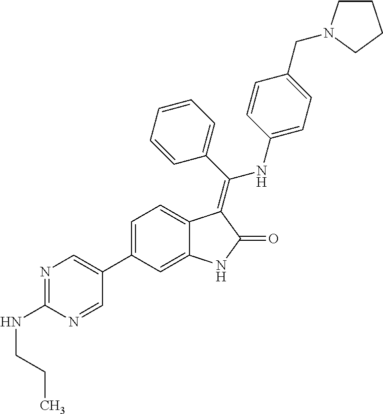 [SMILES: CCCNc1ncc(-c2ccc3c(c2)NC(=O)/C3=C(\Nc2ccc(CN3CCCC3)cc2)c2ccccc2)cn1] |
| 24 | 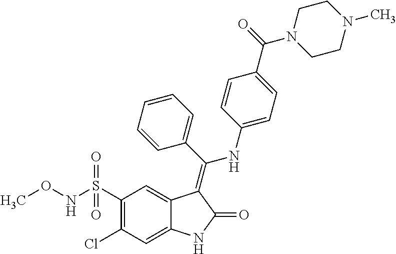 |
| 25 | 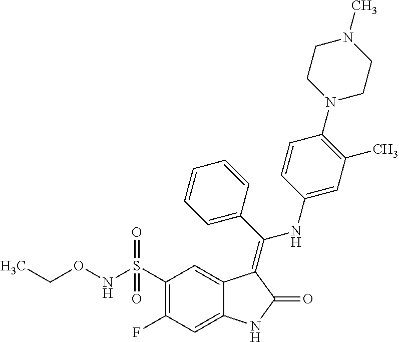 [SMILES: CCONS(=O)(=O)c1cc2c(cc1F)NC(=O)/C2=C(\Nc1ccc(N2CCN(C)CC2)c(C)c1)c1ccccc1] |
| 26 | 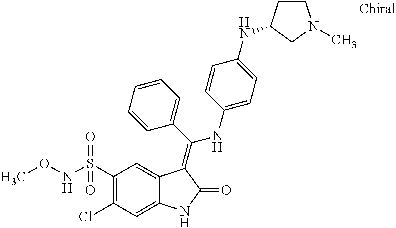 |
| 27 | 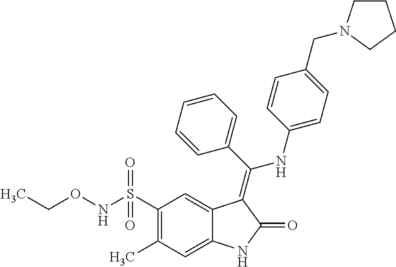 [SMILES: CCONS(=O)(=O)c1cc2c(cc1C)NC(=O)/C2=C(\Nc1ccc(CN2CCCC2)cc1)c1ccccc1] |
| 28 | 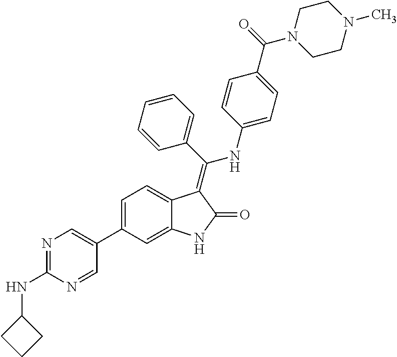 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 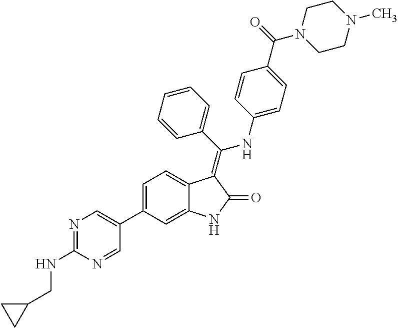 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 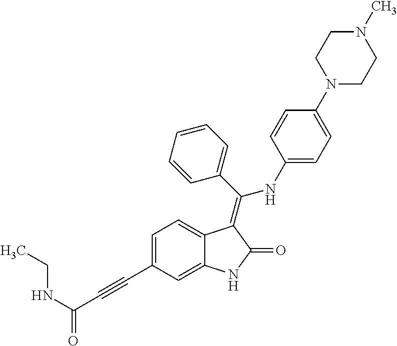 [SMILES: CCNC(=O)C#Cc1ccc2c(c1)NC(=O)/C2=C(\Nc1ccc(N2CCN(C)CC2)cc1)c1ccccc1] |
| 31 | 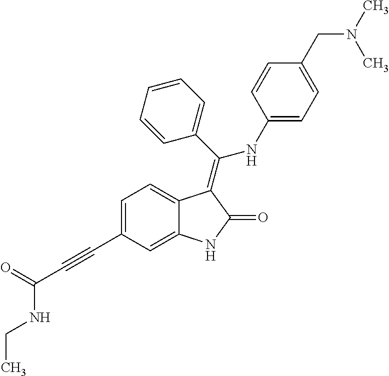 [SMILES: CCNC(=O)C#Cc1ccc2c(c1)NC(=O)/C2=C(\Nc1ccc(CN(C)C)cc1)c1ccccc1] |
| 32 | 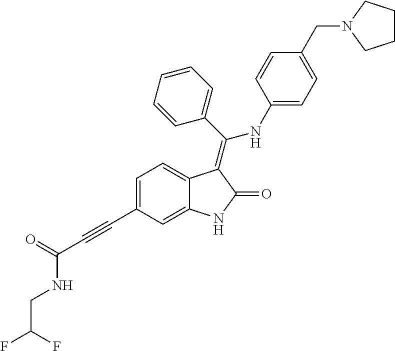 [SMILES: O=C(C#Cc1ccc2c(c1)NC(=O)/C2=C(\Nc1ccc(CN2CCCC2)cc1)c1ccccc1)NCC(F)F] |
| 33 | 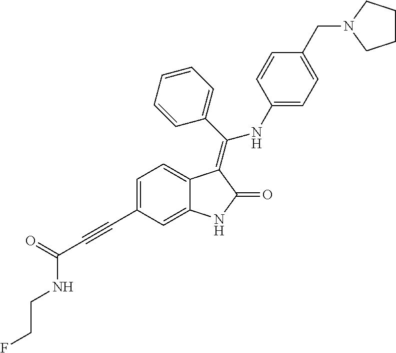 [SMILES: O=C(C#Cc1ccc2c(c1)NC(=O)/C2=C(\Nc1ccc(CN2CCCC2)cc1)c1ccccc1)NCCF] |
| 34 | 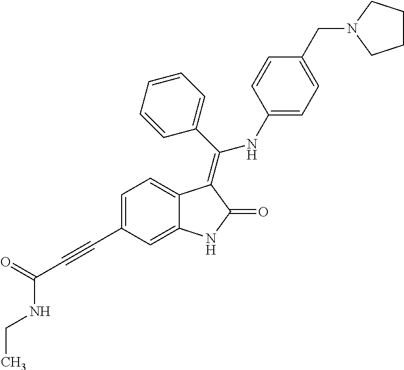 [SMILES: CCNC(=O)C#Cc1ccc2c(c1)NC(=O)/C2=C(\Nc1ccc(CN2CCCC2)cc1)c1ccccc1] |
| 35 | 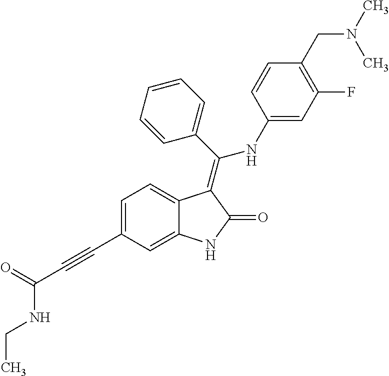 [SMILES: CCNC(=O)C#Cc1ccc2c(c1)NC(=O)/C2=C(\Nc1ccc(CN(C)C)c(F)c1)c1ccccc1] |
| 36 | 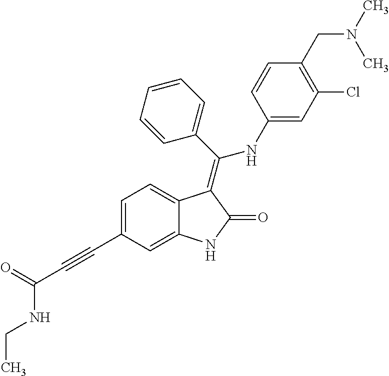 [SMILES: CCNC(=O)C#Cc1ccc2c(c1)NC(=O)/C2=C(\Nc1ccc(CN(C)C)c(Cl)c1)c1ccccc1] |

In another embodiment (embodiment B), the Aurora kinase inhibitor is selected from the group consisting of Barasertib (AZD-1152), AT-9283 1-cyclopropyl-3-[3-(5-morpholin-4-ylmethyl-1H-benzoimidazol-2-yl)-1H-pyrazol-4-yl]urea (cf. WO 2006/070195, Example 24), MLN-8237 4-{[9-chloro-7-(2-fluoro-6-methoxyphenyl)-5H-pyrimido[5,4-d][2]benzazepin-2-yl]amino}-2-methoxybenzoic acid (cf. WO 2008/063525, Example 1), and AS703569/R763 (1R,2R,3S,4S)—N4-(3-aminocarbonylbicyclo[2.2.1]hept-5-ene-2-yl)-5-fluoro-N2-[(3-methyl-4-(4-methylpiperazin-1-yl)]phenyl-2,4-pyrimidinediamine (cf. WO 2005/118544), optionally in the form of its prodrugs and the tautomers and pharmaceutically acceptable salts thereof.

The Aurora kinase inhibitors mentioned herein, methods for their preparation and uses are disclosed in the documents indicated herein. For details, e.g. on a process to manufacture, to formulate or to use such a compound or a salt thereof, reference is thus made to the respective document.

According to the instant invention the cell signalling and/or angiogenesis inhibitor and the Aurora kinase inhibitor can be administered in a single formulation or in two separate formulations. Consequently, in one preferred embodiment the invention relates to pharmaceutical compositions comprising both a cell signalling and/or angiogenesis inhibitor and an Aurora kinase inhibitor.

In another preferred embodiment the invention relates to a kit comprising a first pharmaceutical composition which comprises a cell signalling and/or angiogenesis inhibitor, and a second pharmaceutical composition which comprises an Aurora kinase inhibitor.

The instant invention is furthermore directed to a method for the treatment of oncological and fibrotic diseases which comprises administering to a host in need of such treatment a therapeutically effective amount of a cell signalling and/or angiogenesis inhibitor and an Aurora kinase inhibitor.

In a particular embodiment, the pharmaceutical combinations, compositions, methods and uses according to this invention refer to a combination of an angiogenesis inhibitor, which is BIBF 1120, and an Aurora kinase inhibitor, which is selected from compounds 1 to 36 of Table i.

In another particular embodiment, the pharmaceutical combinations, compositions, methods and uses according to this invention refer to a combination of a cell signalling inhibitor, which is BIBW-2992, and an Aurora kinase inhibitor, which is selected from compounds 1 to 36 of Table i.

Within the context of the invention, compound 1 is optionally applied in the form of the tautomers and pharmaceutically acceptable salts thereof. Pharmaceutically acceptable salts are preferably selected from the group consisting of hydrochloride, hydrobromide, hydriodide, hydrosulphate, hydrophosphate, hydromethanesulphonate, hydroethanesulphonate, hydronitrate, hydromaleate, hydroacetate, hydrobenzoate, hydrocitrate, hydrofumarate, hydrotartrate, hydrolactate, hydroxalate, hydrosuccinate, hydrobenzoate and hydro-p-toluenesulphonate, preferably hydrochloride, hydrobromide, hydroethanesulphonate, hydrosulphate, hydrophosphate, hydromaleate, hydrofumarate and hydromethanesulphonate. In a particularly preferred embodiment compound 1 is applied as its hydroethanesulphonate (1a) depicted below

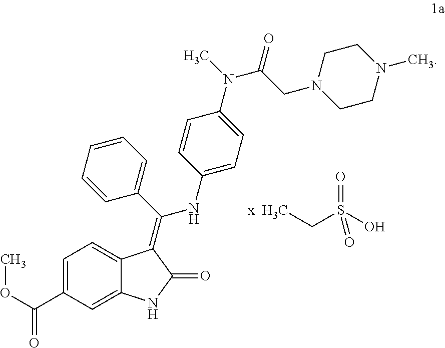

Within the context of this invention the particularly preferred salt of formula 1a is optionally also referred to as the monoethanesulphonate of compound of formula 1. The present invention includes the use of the solvates and hydrates of the salts of the compound 1.

Unless otherwise noted, kinase inhibitors mentioned herein include single kinase inhibitors, which inhibit specifically one kinase and/or one kinase isoform, or multikinase inhibitors, which inhibit two or more kinases and/or two or more kinase isoforms (e.g. dual or triple kinase inhibitors or pan-kinase inhibitors).

Depending on the disease diagnosed, improved treatment outcomes may be obtained if at least one active ingredient of this invention (e.g. angiogenesis inhibitor and/or Aurora kinase inhibitor) is combined with one or more other active substances customary for the respective diseases, such as e.g. one or more active substances selected from among the other anticancer agents, especially those chemotherapeutic agents mentioned herein. Such a combined treatment may be given as a free combination of the substances or in the form of a fixed combination, including kit-of-parts. Pharmaceutical formulations of the combination components needed for this may either be obtained commercially as pharmaceutical compositions or may be formulated by the skilled man using conventional methods.

Whereas the main focus of the invention is directed to the combination of an angiogenesis inhibitor and an Aurora kinase inhibitor, either an angiogenesis inhibitor or a Aurora kinase inhibitor may also be successfully administered in conjunction, with one or more other chemotherapeutic agents, such as e.g. with an inhibitor of the erbB1 receptor (EGFR) and erbB2 (Her2/neu) receptor tyrosine kinases, particularly BIBW-2992. In a particular embodiment the combination of an angiogenesis inhibitor (especially BIBF 1120 or a salt thereof) and an Aurora kinase inhibitor is administered together with the compound of formula 3 (herein referred to as BIBW-2992)

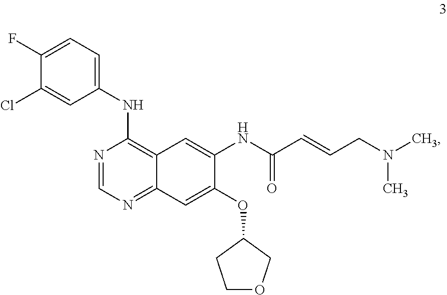 [SMILES: CN(C)C/C=C/C(=O)Nc1cc2c(Nc3ccc(F)c(Cl)c3)ncnc2cc1O[C@H]1CCOC1]

optionally in the form of the tautomers and pharmaceutically acceptable salts thereof.

The compound of formula 3 is a potent and selective dual inhibitor of erbB 1 receptor (EGFR) and erbB2 (Her2/neu) receptor tyrosine kinases. Furthermore, 3 was designed to covalently bind to EGFR and HER2 thereby irreversibly inactivating the receptor molecule it has bound to. This compound 3, salts thereof such as the dimaleate salt, their preparation as well as pharmaceutical formulations comprising 3 or a salt thereof, indications to be treated with 3 and combinations including 3 are disclosed in WO 02/50043, WO 2005/037824, WO 2007/054550 and WO 2007/054551.

Other chemotherapeutic agents which may be administered in conjunction with the active ingredients of this invention (angiogenesis inhibitor and/or Aurora kinase inhibitor) may be selected from the following:

(i) alkylating or carbamylating agents, such as for example nitrogen mustards (with bis-(2-chlorethyl) grouping) such as e.g. cyclophosphamide (CTX, e.g. Cytoxan, Cyclostin, Endoxan), chlorambucil (CHL, e.g. Leukeran), ifosfamide (e.g. Holoxan) or melphalan (e.g. Alkeran), alkyl sulfonates such as e.g. busulphan (e.g. Myleran), mannosulphan or treosulphan, nitrosoureas such as e.g. streptozocin (e.g. Zanosar) or chloroethylnitrosoureas CENU like carmustine BCNU or lomustine CCNU, hydrazines such as e.g. procarbazine, triazenes/imidazotetrazines such as e.g. decarbazine or temozolomide (e.g. Temodar), or ethylenimines/aziridines/methylmelamines such as e.g. mitomycin C, thiotepa or altretamine, or the like;
(ii) platinum derivatives, such as for example cisplatin (CisP, e.g. Platinex, Platinol), oxaliplatin (e.g. Eloxatin), satraplatin or carboplatin (e.g. Carboplat), or the like;
(iii) antimetabolites, such as for example folic acid antagonists such as e.g. methotrexate (MTX, e.g. Farmitrexat), raltitrexed (e.g. Tomudex), edatrexate or pemetrexed (e.g. Alimta), purine antagonists such as e.g. 6-mercaptopurine (6 MP, e.g. Puri-Nethol), 6-thioguanine, pentostatin, cladribine, clofarabine or fludarabine (e.g. Fludara), or pyrimidine antagonists such as e.g. cytarabine (Ara-C, e.g. Alexan, Cytosar), floxuridine, 5-fluorouracil (5-FU) alone or in combination with leucovorin, tegafur, 5-azacytidine (e.g. Vidaza), capecitabine (e.g. Xeloda), decitabine (e.g. Dacogen) or gemcitabine (e.g. Gemzar), or the like;
(iv) antitumor/cyctotoxic antibiotics, such as for example anthracyclines such as e.g. daunorubicin including its hydrochloride salt (including liposomal formulation), doxorubicin including its hydrochloride and citrate salt (e.g. Adriblastin, Adriamycin, including liposomal formulation like Doxil or Caelyx), epirubicin or idarubicin including its hydrochloride salt (e.g. Idamycin), anthracenediones such as e.g. mitoxantrone (e.g. Novantrone), or streptomyces such as e.g. bleomycin, mitomycin or actinomycin D/dactinomycin, or the like;
(v) topoisomerase (including I and II) inhibitors, such as e.g. for example camptothecin and camptothecin analogues such as e.g. irinotecan (e.g. Camptosar) including its hydrochloride, topotecan (e.g. Hycamtin), rubitecan or diflomotecan, epipodophyllotoxins such as e.g. etoposide (e.g. Etopophos) or teniposide, anthracyclines (see above), mitoxantrone, losoxantrone or actinomycin D, or amonafide, or the like;
(vi) microtubule interfering agents, such as for example vinca alkaloids such as e.g. vinblastine (including its sulphate salt), vincristine (including its sulphate salt), vindesine or vinorelbine (including its tartrate salt), taxanes (taxoids) such as e.g. docetaxel (e.g. Taxotere), paclitaxel (e.g. Taxol) or analogues, derivatives or conjugates thereof (e.g. larotaxel), or epothilones such as e.g. epothilone B (patupilone), azaepothilone (ixabepilone), ZK-EPO (sagopilone) or KOS-1584 or analogues, derivatives or conjugates thereof, or the like;
(vii) hormonal therapeutics, such as for example anti-androgens such as e.g. flutamide, nilutamide or bicalutamide (casodex), anti-estrogens such as e.g. tamoxifen, raloxifene or fulvestrant, LHRH agonists such as e.g. goserelin, leuprolide, buserelin or triptolerin; GnRH antagonists such as e.g. abarelix or degarelix; aromatase inhibitors such as e.g. steroids (e.g. exemestane or formestane) or non-steroids (e.g. letrozole, fadrozole or anastrozole).

The therapeutic combination or combined treatment of this invention may further involve or comprise surgery and/or radiotherapy.

The combination treatment according to the invention is of particular interest in the treatment of oncological diseases.

Preferably the disease is selected from solid tumours, such as urogenital cancers (such as prostate cancer, renal cell cancers, bladder cancers), gynecological cancers (such as ovarian cancers, cervical cancers, endometrial cancers), lung cancer, gastrointestinal cancers (such as non-metastatic or metastatic colorectal cancers, pancreatic cancer, gastric cancer, oesophageal cancers, hepatocellular cancers, cholangiocellular cancers), head and neck cancer (e.g. head and neck squamous cell cancer), malignant glioblastoma, malignant mesothelioma, non-metastatic or metastatic breast cancer (e.g. hormone refractory metastatic breast cancer), malignant melanoma or bone and soft tissue sarcomas, and haematologic neoplasias, such as multiple myeloma, acute myelogenous leukemia, chronic myelogenous leukemia, myelodysplastic syndrome and acute lymphoblastic leukemia. In a preferred embodiment, the disease is non small cell lung cancer (NSCLC), breast cancer (e.g. hormone refractory metastatic breast cancer), head and neck cancer (e.g. head and neck squamous cell cancer), malignant glioblastoma, metastatic colorectal cancers, hormone sensitive or hormone refractory prostate cancer, colorectal cancer, ovarian cancer, hepatocellular cancer, renal cell cancer, soft tissue sarcoma, or small cell lung cancer.

Also, the following cancer diseases can be treated with the combination according to the invention, without, however, being restricted thereto: brain tumours, such as acoustic neurinoma, astrocytomas such as piloid astrocytomas, fibrillary astrocytoma, protoplasmic astrocytoma, gemistocytic astrocytoma, anaplastic astrocytoma and glioblastomas, brain lymphomas, brain metastases, hypophyseal tumour such as prolactinoma, HGH (human growth hormone) producing tumour and ACTH-producing tumour (adrenocorticotrophic hormone), craniopharyngiomas, medulloblastomas, meningiomas and oligodendrogliomas; nerve tumours (neoplasms) such as tumours of the vegetative nervous system such as neuroblastoma sympathicum, ganglioneuroma, paraganglioma (phaeochromocytoma and chromaffinoma) and glomus caroticum tumour, tumours in the peripheral nervous system such as amputation neuroma, neurofibroma, neurinoma (neurilemoma, schwannoma) and malignant schwannoma, as well as tumours in the central nervous system such as brain and spinal cord tumours; intestinal cancer such as rectal carcinoma, colon carcinoma, anal carcinoma, small intestine tumours and duodenal tumours; eyelid tumours such as basalioma or basal cell carcinoma; pancreatic gland cancer or pancreatic carcinoma; bladder cancer or bladder carcinoma; lung cancer (bronchial carcinoma) such as small-cell bronchial carcinomas (oat cell carcinomas) and non-small-cell bronchial carcinomas such as squamous epithelium carcinomas, adenocarcinomas and large-cell bronchial carcinomas; breast cancer such as mammary carcinoma, such as infiltrating ductal carcinoma, colloid carcinoma, lobular invasive carcinoma, tubular carcinoma, adenoid cystic carcinoma, and papillary carcinoma; non-Hodgkin's lymphomas (NHL) such as Burkitt's lymphoma, low-malignancy non-Hodkgin's lymphomas (NHL) and mucosis fungoides; uterine cancer or endometrial carcinoma or corpus carcinoma; CUP syndrome (cancer of unknown primary); ovarian cancer or ovarian carcinoma such as mucinous, endometrial or serous cancer; gall bladder cancer; bile duct cancer such as Klatskin's tumour; testicular cancer such as seminomas and non-seminomas; lymphoma (lymphosarcoma) such as malignant lymphoma, Hodgkin's disease, non-Hodgkin's lymphomas (NHL) such as chronic lymphatic leukaemia, hair cell leukaemia, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as vocal cord tumours, supraglottal, glottal and subglottal laryngeal tumours; bone cancer such as osteochondroma, chondroma, chrondoblastoma, chondromyxoidfibroma, osteoma, osteoid-osteoma, osteoblastoma, eosinophilic granuloma, giant cell tumour, chondrosarcoma, osteosarcoma, Ewing's sarcoma, reticulosarcoma, plasmocytoma, fibrous dysplasia, juvenile bone cyst and aneurysmatic bone cyst; head/neck tumours such as tumours of the lips, tongue, floor of the mouth, oral cavity, gingiva, pallet, salivary glands, pharynx, nasal cavities, paranasal sinuses, larynx and middle ear; liver cancer such as liver cell carcinoma or hepatocellular carcinoma (HCC); leukaemias, such as acute leukaemias, such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or stomach carcinoma such as papillary, tubular and mucinous adenocarcinoma, signet ring cell carcinoma, adenoid squamous cell carcinoma, small-cell carcinoma and undifferentiated carcinoma; melanomas such as superficially spreading, nodular malignant lentigo and acral lentiginous melanoma; renal cancer, such as kidney cell carcinoma or hypernephroma or Grawitz's tumour; oesophageal cancer or oesophageal carcinoma; cancer of the penis; prostate cancer; pharyngeal cancer or pharyngeal carcinomas such as nasopharyngeal carcinomas, oropharyngeal carcinomas and hypopharyngeal carcinomas; retinoblastoma; vaginal cancer or vaginal carcinoma; squamous epithelium carcinomas, adeno carcinomas, in situ carcinomas, malignant melanomas and sarcomas; thyroid gland carcinomas such as papillary, follicular and medullary thyroid gland carcinoma, and also anaplastic carcinomas; spinalioma, prickle cell carcinoma and squamous epithelium carcinoma of the skin; thymomas, urethral cancer and vulvar cancer.

In another embodiment the combination according to the invention is useful for the prevention or treatment of a specific fibrotic disease selected from the group consisting of: Fibrosis and remodeling of lung tissue in chronic obstructive pulmonary disease (COPD), chronic bronchitis, and emphysema; Lung fibrosis and pulmonary diseases with a fibrotic component including but not limited to idiopathic pulmonary fibrosis (IPF), giant cell interstitial pneumonia (GIP), sarcodosis, cystic fibrosis, respiratory distress syndrome (ARDS), granulomatosis, silicosis, drug-induced lung fibrosis (for example, induced by drugs such as bleomycin, bis-chloronitrosourea, cyclophosphamide, amiodarone, procainamide, penicillamine, gold or nitrofurantoin), silicosis, asbestosis, systemic scleroderma; Fibrosis and remodeling in asthma; Fibrosis in rheumatoid arthritis; Virally induced hepatic cirrhosis, for example hepatitis C; Radiation-induced fibrosis; Restenosis, post angioplasty; Renal disorders including chronic glomerulonephritis, renal fibrosis in patients receiving cyclosporine and renal fibrosis due to high blood pressure; Diseases of the skin with a fibrotic component including but not limited to, scleroderma, sarcodosis, systemic lupus erythematosus; Excessive scarring. In an embodiment, the disease is idiopathic pulmonary fibrosis (IPF).

A particular disease amenable to the combination treatment according to the invention is lung cancer (such as e.g. non small cell lung cancer (NSCLC)).

The dosage of the active ingredients in the combinations and compositions in accordance with the present invention may be varied, although the amount of the active ingredients, particularly cell signalling and/or angiogenesis inhibitor and the Aurora kinase inhibitor shall be such that a suitable dosage form is obtained. Hence, the selected dosage and the selected dosage form shall depend on the desired therapeutic effect, the route of administration and the duration of the treatment. Suitable dosage ranges for the combination are from the maximal tolerated dose for the single agent to lower doses, e.g. to one tenth of the maximal tolerated dose.

Preferably, between 5 and 1000 mg, particularly preferably 10 to 500 mg of the compound of formula 1 are administered once or several times per day in order to implement the medication according to the invention. Particularly preferably, 25-300 mg, more preferably 50-200 mg of compound 1 are administered once or twice daily, preferably twice daily.

The dosage of Aurora kinase inhibitor (particularly of embodiment A) for intravenous use is from 1-1000 mg per hour, preferably between 5 and 500 mg per hour. However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The foregoing doses are based on the free bases of the compounds 1 and the Aurora kinase inhibitor. If these compounds are applied in the form of their pharmaceutically acceptable salts the amount of the appropriate salt can easily be calculated by the skilled artisan.

For the combination therapy according to the invention the cell signalling and/or angiogenesis inhibitor and the Aurora kinase inhibitor may be administered separately (which implies that they are formulated separately) or together (which implies that they are formulated together). Hence, the administration of one element of the combination of the present invention may be prior to, concurrent to, or subsequent to the administration of the other element of the combination. Preferably the cell signalling and/or angiogenesis inhibitor and the Aurora kinase inhibitor are administered in different formulations.

As mentioned hereinbefore, the invention relates to pharmaceutical compositions comprising a cell signalling and/or angiogenesis inhibitor (especially compound 1 or 3, or a tautomer or pharmaceutically acceptable salt thereof) and also an Aurora kinase inhibitor (especially compounds 1-36 listed above, or a tautomer or pharmaceutically acceptable salt thereof). Consequently, if not indicated otherwise throughout the disclosure of this patent application a reference to a combination of a cell signalling and/or angiogenesis inhibitor and an Aurora kinase inhibitor is to be understood referring especially to a combination of compound 1 or 3, or a tautomer or pharmaceutically acceptable salt thereof, and an Aurora kinase inhibitor (especially compounds 1-36 listed above, or a tautomer or pharmaceutically acceptable salt thereof).

The cell signalling and/or angiogenesis inhibitor and the Aurora kinase inhibitor may be administered by oral (including buccal or sublingual), enterical, parenteral (e.g., intramuscular, intraperitoneal, intravenous, transdermal or subcutaneous injection, or implant), nasal, vaginal, rectal, or topical (e.g. ocular eyedrops) routes of administration and may be formulated, alone or together, in suitable dosage unit formulations containing conventional non-toxic pharmaceutically acceptable carriers, adjuvants and vehicles appropriate for each route of administration.

In a preferred embodiment the cell signalling and/or angiogenesis inhibitor of the combination in accordance with the invention is administered orally, enterically, transdermally, intravenously, peritoneally or by injection, preferably orally. In another preferred embodiment the Aurora kinase inhibitor of the combination is preferably administered orally as well. In yet another preferred embodiment the Aurora kinase inhibitor of the combination is preferably administered intravenously (e.g. ranging from bolus injection to prolonged infusion), preferably by infusion.

Continuous administration such as by intravenous infusion of a (liquid) solution or suspension for infusion, which comprises one or more active agents, e.g. from a infusion pump, bag or reservoir (which may be optionally implanted or portable) is also contemplated.

The pharmaceutical compositions for the administration of the cell signalling and/or angiogenesis inhibitor and Aurora kinase inhibitor of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients (cell signalling and/or angiogenesis inhibitor and Aurora kinase inhibitor), separately or together, that are suitable for oral administration may be in the form of discrete units such as hard or soft capsules, tablets, troches or lozenges, each containing a predetermined amount of the active ingredients, or in the form of a dispersible powder or granules, or in the form of a solution or a suspension in an aqueous liquid or non-aqueous liquid, or in the form of syrups or elixirs, or in the form of an oil-in-water emulsion or a water-in-oil emulsion.

Dosage forms intended for oral use may be prepared according to any method known to the art for the manufacture of pharmaceutical formulations and such compositions.

The excipients used may be, for example: (a) inert diluents; (b) granulating and disintegrating agents; (c) binding agents; and (d) lubricating agents.

In some cases, formulations for oral use may be in the form of hard gelatin or HPMC (hydroxypropylmethylcellulose) capsules wherein the active ingredients (cell signalling and/or angiogenesis inhibitor and Aurora kinase inhibitor), separately or together, is mixed with an inert solid diluent, or dispensed via a pellet formulation. They may also be in the form of soft gelatin capsules wherein the active ingredient is mixed with water or an oil medium.

The tablets, capsules or pellets may be uncoated or they may be coated by known techniques, for example to delay disintegration and absorption in the gastrointestinal tract and thereby provide a delayed action or sustained action over a longer period. For example, a normal tablet coating material or a time delay material or sustained release material may be employed. The tablets may also comprise several layers.

Liquid dosage forms for oral administration in accordance with the present invention include pharmaceutically acceptable emulsions, solutions, suspensions, syrups, and elixirs containing inert diluents commonly used in the art, such as water. Besides such inert diluents, compositions can also include adjuvants, such as wetting agents, emulsifying, thickeners and suspending agents, and sweetening, flavoring, perfuming and preserving agents.

Aqueous suspensions in accordance with the present invention normally contain the active materials (cell signalling and/or angiogenesis inhibitor and Aurora kinase inhibitor), separately or together, in admixture with excipients suitable for the manufacture of aqueous suspensions.

Such excipients may be (a) suspending agents; (b) dispersing or wetting agents which may be (b.1) a naturally-occurring phosphatide, (b.2) a condensation product of an alkylene oxide with a fatty acid, (b.3) a condensation product of ethylene oxide with a long chain aliphatic alcohol, (b.4) a condensation product of ethylene oxide with a partial ester derived from a fatty acid and a hexitol, or (b.5) a condensation product of ethylene oxide with a partial ester derived from a fatty acid and a hexitol anhydride.

The aqueous suspensions may also contain: one or more preservatives; one or more coloring agents; one or more flavoring agents; and one or more sweetening agents.

Oily suspensions in accordance with the present invention may be formulated by suspending the active ingredients (cell signalling and/or angiogenesis inhibitor and Aurora kinase inhibitor), separately or together, in a vegetable oil. The oily suspensions may contain a thickening agent. Sweetening agents and flavoring agents may be added to provide a palatable oral preparation. These compositions may be prepared by the addition of an antioxidant.

Dispersible powders and granules are suitable formulations for the preparation of an aqueous suspension in accordance with the present invention. In these formulations the active ingredients (cell signalling and/or angiogenesis inhibitor and Aurora kinase inhibitor) are present, separately or together, in admixture with a dispersing or wetting agent, a suspending agent and one or more preservatives. Suitable examples of dispersing or wetting agents, suspending agents and preservatives are those already mentioned hereinbefore. Additional excipients such as, for example, sweetening, flavouring and colouring agents may also be present. Suitable examples of excipients are those already mentioned hereinbefore.

The pharmaceutical compositions of the invention may also be in the form of oil-in-water emulsions. The oily phase may be a vegetable oil or a mineral oil or a mixture thereof.

Suitable emulsifying agents may be (a) naturally-occurring gums, (b) naturally-occurring phosphatides, (c) esters or partial esters derived from fatty acids and hexitol anhydrides, (d) condensation products of said partial esters with ethylene oxide. The emulsions may also contain sweetening and flavouring agents.

Syrups and elixirs in accordance with the present invention may be formulated with sweetening agents. Such formulations may also contain a preservative and flavoring and coloring agents.

Preparations for parenteral administration according to the present invention containing a cell signalling and/or angiogenesis inhibitor and an Aurora kinase inhibitor, separately or together, include sterile aqueous, semi-aqueous, non-aqueous, oily or mixed solvent systems injection or infusion solutions, suspensions or emulsions.

The pharmaceutical compositions containing an angiogenesis inhibitor and a cell signalling and/or Aurora kinase inhibitor, separately or together, may be in the form of steril isotonic aqueous or semi-aqueous injection or infusion solutions or suspensions, or concentrates or lyophilisates for such solutions or suspensions to be produced prior to use, e.g. by diluting with isotonic aqueous medium.

The pharmaceutical compositions containing a cell signalling and/or angiogenesis inhibitor and an Aurora kinase inhibitor, separately or together, may be in the form of a sterile injectable or infusionable aqueous or oleagenous suspension or solution. The suspension may be formulated according to known methods using those suitable dispersing or wetting agents and suspending agents which have been mentioned hereinbefore. A suitable sterile injectable or infusionable preparation may also be a sterile injectable or infusionable solution or suspension in a non toxic parenterally-acceptable diluent or solvent. Examples of acceptable vehicles and solvents that may be employed are water, dextrose solution, Ringer's solution and an isotonic sodium chloride solution. In addition, sterile, fixed oils may conventionally be employed as a solvent or suspending medium. For this purpose any bland fixed oil may be employed, including synthetic mono- or diglycerides. In addition, fatty acids may find use in the preparation of injectables or infusionables. Non-aqueous solvents or vehicles comprised in such preparations in accordance with the present invention may include e.g. propylene glycol, polyethylene glycol, mono- or polyfunctional alcohols, vegetable oils, or injectable or infusionable organic esters. Such dosage forms may also contain adjuvants such as preserving, wetting, emulsifying, dispersing or pH-adjusting agents.

They may be sterilized by, for example, by filtration through a bacteria-retaining filter, by incorporating sterilizing agents into the compositions, by irradiating the compositions, or by heating the compositions. They may also be manufactured in the form of sterile solid compositions which can be reconstituted in sterile water, or some other sterile injectable or infusionable medium immediately before use.

Solutions for injection and infusion are prepared in the usual way, e.g. with the addition of one or more suitable aqueous and/or non-aqueous solvents (e.g. isotonic agents) and, optionally, preservatives, stabilisers, emulsifiers, dispersants and/or pH-adjusting agents, whilst if water is used as the diluent, for example, organic solvents may optionally be used as solvating agents or dissolving aids, and transferred into injection vials or ampoules or infusion bottles. For example, by a process comprising the addition of one or more suitable organic solvents, such as e.g. mono- or polyfunctional alcohols, polypropylene glycol or polyethylene glycol, and of a pH-adjusting agent an organic concentrate for solution for infusion can be prepared, which may be optionally lyophilized. Before application to the patient, such organic concentrate is diluted with an appropriate infusion solution (e.g. aqueous dextrose solution 5%) to provide the applicable form.

The cell signalling and/or angiogenesis inhibitor and Aurora kinase inhibitor of the combination of this invention may also be administered in the form of suppositories for rectal administration. Such compositions can be prepared by mixing the active ingredient with a suitable non-irritating excipient which is solid at ordinary temperatures but liquid at the rectal temperature and will therefore melt in the rectum to release the active ingredient.

Compositions for buccal, nasal or sublingual administration in accordance with the present invention may be prepared with standard excipients well known in the art.

For topical administration, the elements (a cell signalling and/or angiogenesis inhibitor and an Aurora kinase inhibitor) of the combination of this invention may be formulated, separately or together, in liquid or semi-liquid preparations. Examples of suitable preparations are: liniments, lotions, applications; oil-in-water or water-in-oil emulsions such as creams, ointments, jellies or pastes, including tooth-pastes; solutions or suspensions such as drops.

In a preferred embodiment, the active ingredient 1 or a pharmaceutically acceptable salt thereof is formulated in the form of a capsule such as for example a hard gelatin or a hydroxypropylmethylcellulose (HPMC) capsule, comprising a capsule shell and a capsule formulation, wherein the capsule formulation comprises a suspension of the active ingredient 1 or a pharmaceutically acceptable salt thereof, preferably a viscous suspension comprising a carrier and a thickener, more preferably a viscous suspension in which the carrier is a lipid (lipophilic) carrier.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### EXPERIMENTAL PART

### A) Preferred Examples of Dosage Forms Comprising 1

The tables below show pharmaceutical compositions for 1.

The active substance in all the Examples is 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate.

### Example 1

Soft gelatin capsule containing 50 mg of active substance

| | | Formulation | Formulation | Formulation |
|---|---|---|---|---|
| | | A | B | C |
| | | mg per | mg per | mg per |
| Ingredients | Function | capsule | capsule | capsule |
| Active | Active | 60.20 | 60.20 | 60.20 |
| Substance* | Ingredient | | | |
| Triglycerides, | Carrier | 40.95 | 53.70 | 54.00 |
| Medium-chain | | | | |
| Hard fat | Thickener | 38.25 | 25.50 | 25.50 |
| Lecithin | Wetting | 0.60 | 0.60 | 0.30 |
| | agent/ | | | |
| | Glidant | | | |
| Gelatin | Film- | 72.25 | 72.25 | 72.25 |
| | former | | | |
| Glycerol 85% | Plasticizer | 32.24 | 32.24 | 32.24 |
| Titanium | Colorant | 0.20 | 0.20 | 0.20 |
| dioxide | | | | |
| Iron oxide A | Colorant | 0.32 | 0.32 | 0.32 |
| Iron oxide B | Colorant | 0.32 | 0.32 | 0.32 |
| Total Capsule | | 245.33 | 245.33 | 245.33 |
| Weight | | | | |

*The FIGURES refer to the amount of ethanesulfonate salt (dry basis) equivalent to the labeled amount of the free base

### Example 1a

### Soft Gelatin Capsule Containing 75 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active | Active | 90.3 | 90.3 | 90.3 |
| Substance* | Ingredient | | | |
| Triglycerides, | Carrier | 61.425 | 80.55 | 80.1 |
| Medium-chain | | | | |
| Hard fat | Thickener | 57.375 | 38.25 | 38.25 |
| Lecithin | Wetting | 0.9 | 0.9 | 1.35 |
| | agent/ | | | |
| | Glidant | | | |
| Gelatin | Film- | 107.11 | 107.11 | 107.11 |
| | former | | | |
| Glycerol 85% | Plasticizer | 46.84 | 46.84 | 46.84 |
| Titanium | Colorant | 0.35 | 0.35 | 0.35 |
| dioxide | | | | |
| Iron oxide A | Colorant | 0.058 | 0.058 | 0.058 |
| Iron oxide B | Colorant | 0.16 | 0.16 | 0.16 |
| Total Capsule | | 364.518 | 364.518 | 364.518 |
| Weight | | | | |

*The FIGURES refer to the amount of ethanesulfonate salt (dry basis) equivalent to the labeled amount of the free base

### Example 2

### Soft Gelatin Capsule Containing 100 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active | Active | 120.40 | 120.40 | 120.40 |
| Substance* | Ingredient | | | |
| Triglycerides, | Carrier | 81.90 | 107.40 | 106.8 |
| Medium-chain | | | | |
| Hard fat | Thickener | 76.50 | 51.00 | 51.00 |
| Lecithin | Wetting | 1.20 | 1.20 | 1.80 |
| | agent/ | | | |
| | Glidant | | | |
| Gelatin | Film- | 111.58 | 111.58 | 111.58 |
| | former | | | |
| Glycerol 85% | Plasticizer | 48.79 | 48.79 | 48.79 |
| Titanium | Colorant | 0.36 | 0.36 | 0.36 |
| dioxide | | | | |
| Iron oxide A | Colorant | 0.06 | 0.06 | 0.06 |
| Iron oxide B | Colorant | 0.17 | 0.17 | 0.17 |
| Total Capsule | | 440.96 | 440.96 | 440.96 |
| Weight | | | | |

*The FIGURES refer to the amount of ethanesulfonate salt (dry basis) equivalent to the labeled amount of the free base

### Example 3

### Soft Gelatin Capsule Containing 125 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active Substance* | Active | 150.50 | 150.50 | 150.50 |
| | Ingredient | | | |
| Triglycerides, | Carrier | 102.375 | 134.25 | 133.5 |
| Medium-chain | | | | |
| Hard fat | Thickener | 95.625 | 63.75 | 63.75 |
| Lecithin | Wetting | 1.50 | 1.50 | 2.25 |
| | agent/ | | | |
| | Glidant | | | |
| Gelatin | Film- | 142.82 | 142.82 | 142.82 |
| | former | | | |
| Glycerol 85% | Plasticizer | 62.45 | 62.45 | 62.45 |
| Titanium dioxide | Colorant | 0.47 | 0.47 | 0.47 |
| Iron oxide A | Colorant | 0.08 | 0.08 | 0.08 |
| Iron oxide B | Colorant | 0.22 | 0.22 | 0.22 |
| Total Capsule | | 556.04 | 556.04 | 556.04 |
| Weight | | | | |

*The FIGURES refer to the amount of ethanesulfonate salt (dry basis) equivalent to the labeled amount of the free base

### Example 4

### Soft Gelatin Capsule Containing 150 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active Substance* | Active | 180.60 | 180.60 | 180.60 |
| | Ingredient | | | |
| Triglycerides, | Carrier | 122.85 | 161.10 | 160.20 |
| Medium-chain | | | | |
| Hard fat | Thickener | 114.75 | 76.50 | 76.50 |
| Lecithin | Wetting | 1.80 | 1.80 | 2.70 |
| | agent/ | | | |
| | Glidant | | | |
| Gelatin | Film- | 142.82 | 142.82 | 142.82 |
| | former | | | |
| Glycerol 85% | Plasticizer | 62.45 | 62.45 | 62.45 |
| Titanium dioxide | Colorant | 0.47 | 0.47 | 0.47 |
| Iron oxide A | Colorant | 0.08 | 0.08 | 0.08 |
| Iron oxide B | Colorant | 0.22 | 0.22 | 0.22 |
| Total Capsule | | 626.04 | 626.04 | 626.04 |
| Weight | | | | |

*The FIGURES refer to the amount of ethanesulfonate salt (dry basis) equivalent to the labeled amount of the free base

### Example 5

Soft Gelatin Capsule Containing 200 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active | Active | 240.80 | 240.80 | 240.80 |
| Substance* | Ingredient | | | |
| Triglycerides, | Carrier | 163.30 | 214.80 | 216.00 |
| Medium-chain | | | | |
| Hard fat | Thickener | 153.50 | 102.00 | 102.00 |
| Lecithin | Wetting | 2.40 | 2.40 | 1.20 |
| | agent/ | | | |
| | Glidant | | | |
| Gelatin | Film- | 203.19 | 203.19 | 203.19 |
| | former | | | |
| Glycerol 85% | Plasticizer | 102.61 | 102.61 | 102.61 |
| Titanium | Colorant | 0.57 | 0.57 | 0.57 |
| dioxide | | | | |
| Iron oxide A | Colorant | 0.90 | 0.90 | 0.90 |
| Iron oxide B | Colorant | 0.90 | 0.90 | 0.90 |
| Total Capsule | | 868.17 | 868.17 | 868.17 |
| Weight | | | | |

*The FIGURES refer to the amount of ethanesulfonate salt (dry basis) equivalent to the labeled amount of the free base

### Example 6

The table below shows further pharmaceutical compositions according to the invention. D, E and F are tablets, G can be compressed to form tablets after hot melt-granulation of the active substance in a heated/cooled high-shear mixer together with Microcrystalline cellulose an Macrogol 6000. After further mixing steps of the obtained granules with the other excipients, tablets are produced on a conventional tablet press. Alternatively it can be directly dispensed as oral granules into sachets.

Tablet D and F may be produced by direct blending of the components and subsequent compression on a conventional tablet press. Alternatively it can be extruded to pellets and filled into a hard capsule.

Tablet E may be produced by wet granulation of the drug substance together with Lactose monohydrate and Microcrystalline cellulose by an aqueous solution of Copovidone. After further blending steps with Crospovidone, Colloidal silica and Magnesium stearate, the tablets are compressed on a conventional tablet press.

| Formulation | D | | E | | F | | G | | H | | I | |
|---|---|---|---|---|---|---|---|---|---|---|---|---|
| Active Substance* | 180.6 | mg | 150.5 | mg | 120.4 | mg | 150.5 | mg | 60.2 | mg | 60.2 | mg |
| Sorbitol | — | | — | | — | | — | | — | | 125.0 | mg |
| Lactose monohydrate | 50.0 | mg | 125.0 | mg | — | | — | | — | | — | |
| Microcrystalline cellulose | — | | 20.0 | mg | 150.0 | mg | 80.0 | mg | — | | 20.0 | mg |
| Calcium phopsphate | 30.0 | mg | — | | 150.0 | mg | — | | — | | — | |
| Soybean Oil | — | | — | | — | | — | | 145.0 | mg | — | |
| Macrogol 6000 | — | | — | | — | | 80.0 | mg | — | | — | |
| Copovidone | 2.0 | mg | 10.0 | mg | — | | — | | — | | — | |
| Sodium starch glycolate | 5.0 | mg | — | | — | | — | | — | | — | |
| Crospovidone | — | | 5.0 | mg | 5.0 | mg | — | | — | | 5.0 | mg |
| Cremophor RH 40 | — | | — | | — | | — | | 20.0 | mg | — | |
| Colloidal silica | 1.0 | mg | 1.0 | mg | 1.0 | mg | — | | 10.0 | mg | 1.0 | mg |
| Solid flavour | — | | — | | — | | 5.0 | mg | — | | 4.0 | mg |
| Magnesium stearate | 4.0 | mg | 4.0 | mg | 4.0 | mg | — | | — | | — | |
| Total | 272.6 | mg | 315.5 | mg | 430.4 | mg | 315.5 | mg | 235.2 | mg | 215.2 | mg |

*The figures refer to the amount of ethanesulfonate salt 1a

Formulation H is prepared as a liquid fillmix of suspended active. After homogenization it is filled either in hard or soft gelatine capsules. Formulation I is an oral powder.

### B) In Vitro Study Results of Aurora Kinase Inhibitor

Compound X, a potent inhibitor of Aurora B kinase (IC₅₀=9 nM) of Table i according to this invention, blocks proliferation in various human cancer cell lines (EC₅₀=2-14 nM) and induces polyploidy, senescence and apoptosis.

Methods.

Compound X was profiled in enzymatic kinase assays as well as in proliferation assays on various human cancer cell lines. Cell cycle status was assessed by DNA content analysis (Cellomics ArrayScan, FACScalibur). Histone H3 phosphorylation was determined by immunofluorescence (Cellomics ArrayScan). Apoptosis was detected by Western blotting for cleaved PARP and microscopic enumeration of DAPI-stained cells showing nuclear fragmentation. Senescent cells were identified by staining for SA-β-Gal activity.

Results.

Compound X inhibited human Aurora B kinase activity with an IC₅₀ value of 9 nM, Aurora A and C kinases with 70 nM and 17 nM, respectively. In a panel of 46 additional kinases representative of the human kinome, Compound X at 1000 nM inhibited 7/46 kinases by more than 50%. EC₅₀ values for inhibition of proliferation of >20 human cancer cell lines were in the range of 2 to 14 nM. In the non-small cell lung cancer cell line NCI-H460, treatment with Compound X resulted in a rapid (<1 h) inhibition of histone H3 phosphorylation. Within 48 h of treatment, the fraction of polyploid cells increased from <5% to >80%, paralleled by a marked increase in cell volume. An increase of cleaved poly (ADP-ribose) polymerase and a concomitant increase in the fraction of cells with nuclear fragmentation from <1% to 7% was observed after 72 h and 96 h of treatment. A pronounced increase of senescent cells from <3% to 25% of the population was observed within 96 h.

### C) In Vivo Study Results of Aurora Kinase Inhibitor

Compound X, an inhibitor of Aurora B kinase of Table i according to this invention, demonstrates potent antitumor activity in multiple cancer models at well-tolerated doses; treated tumors show hallmark of Aurora B inhibition. Continuous infusion over 24 h provides a superior therapeutic index compared with bolus administration.

Methods.

BomTac:NMRI-Foxn1^{nu} mice were grafted subcutaneously with NCI-H460 non-small cell lung carcinoma (mutant KRAS, wild-type p53), HCT 116 colon carcinoma (mutant KRAS, wild-type p53) or BxPC-3 pancreas adenocarcinoma cells (wild-type KRAS, mutant p53). Treatment was initiated when the tumors had reached a volume of ˜50 mm³. BI 811283 was injected intravenously once or twice weekly as a single bolus or b.i.d. Alternatively, the compound was administered once-weekly by a continuous 24 h infusion via subcutaneously implanted osmotic mini-pumps. Multiple dose levels and dosing schedules were evaluated.

Results.

In models of human non-small cell lung cancer, colon carcinoma and pancreas carcinoma, multiple cycles of treatment with Compound X at total weekly doses of 20 to 75 mg/kg resulted in dose-dependent inhibition of tumor growth or tumor regression. Continuous s.c. infusion at 20 mg/kg over 24 h once-weekly was clearly superior to all bolus injection schedules delivering weekly doses up to 75 mg/kg. Furthermore, regression of large tumors (350 mm³) was induced in the HCT 116 colon carcinoma model. Biomarker analyses of HCT 116 tumors revealed that therapeutic doses of Compound X inhibited phosphorylation of histone H3, a direct substrate of Aurora B. Histological examination showed an accumulation of enlarged, multinucleated cells in accordance with the expected mechanism of action.

### Dose- and Schedule-Dependency:

| | | Dose | Weekly dose | | | T/C |
|---|---|---|---|---|---|---|
| Cancer type | Model | [mg/kg] | [mg/kg] | Schedule | Route | [%] |
| Colon carcinoma | HCT 116 | 30 | 60 | (qdx2)x6 | i.v. | 25 |
| | | 15 BID | 60 | (qdx2)x6 | i.v. | 4 |
| | | 37.5 BID | 75 | (q7d)x6 | i.v. | 8 |
| | | 20 | 20 | (q7d)x5 | 24 h cont. inf. | 2 |
| NSCLC | NCI-H460 | 15 BID | 60 | (qdx2)x3 | i.v. | 27 |
| | | 20 | 20 | (q7d)x3 | 24 h cont. inf. | 8 |
| Pancreatic carcinoma | BxPC-3 | 15 BID | 60 | (qdx2)x6 | i.v. | 17 |
| | | 20 | 20 | (q7d)x4 | 24 h cont. inf. | 6 |

Compound X is also potent in AML cancer models (THP-1 with T/C value of 7% and MV-4; 11 with T/C value of 5%).

### D) Preclinical In Vivo Study Results in a Lung Cancer Model

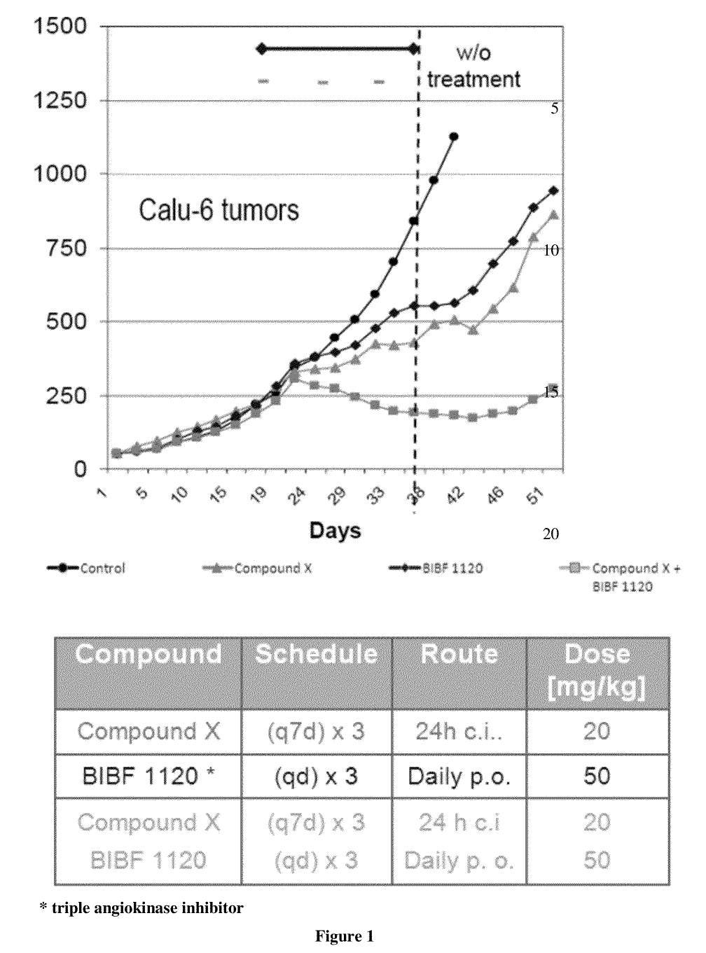

In order to analyse the anti-tumor effects of combining the inhibition of tumor angiogenesis by interfering with the VEGFR signalling cascade with the antitumor activity of inhibiting Aurora kinase B, the following in vivo experiment is performed. Nude mice carrying established subcutaneous Calu-6 xenografts (human NSCLC tumor cell line) are randomized and treated with either the Aurora B kinase inhibitor of this invention Compound X or BIBF 1120 alone or with the combination of both drugs. After termination of treatment the tumors on the control treated mice (black line with circles in FIG. 1) are in average about 1100 mm³ in volume. The results of FIG. 1 show that the combination of Compound X and BIBF 1120 in large tumors (at start of treatment ca. 350 mm³) result in improved antitumor efficacy (lowest green line with squares in FIG. 1) compared to single agent treatments with Compound X (orange line with triangles in FIG. 1) and with BIBF 1120 (blue line with rhombs in FIG. 1), respectively.

Combination treatment with Compound X and BIBF 1120 has a median T/C value of 16% @ d40 compared to 45% @ d40 for Compound X treatment alone and to 50% @ d40 for BIBF 1120 treatment alone.

FIG. 1: Calu-6 NSCLC model, combination BIBF 1120+Compound X, schedule (cf. Drawings; control group: line with circles; BIBF 1120 treated group: line with rhombs; Compound X (AKI) treated group: line with triangles; Combo BIBF 1120+Compound X (AKI) treated group: line with squares).

1. A pharmaceutical composition comprising an angiogenesis inhibitor and an Aurora kinase inhibitor (AKI).

2. The pharmaceutical composition of claim 1, wherein the cell signalling and/or angiogenesis inhibitor is an angiogenesis inhibitor.

3. The pharmaceutical composition of claim 1, wherein the cell signalling and/or angiogenesis inhibitor is a cell signalling inhibitor.

4. The pharmaceutical composition of claim 2, wherein the angiogenesis inhibitor is a compound targeting vascular endothelial growth factor VEGF or the VEGF receptor.

5. The pharmaceutical composition of claim 2, wherein the angiogenesis inhibitor is bevacizumab, aflibercept (VEGF-Trap), vandetanib, cediranib, axitinib, sorafenib, sunitinib, motesanib, vatalanib, pazopanib or BIBF 1120.

6. The pharmaceutical composition of claim 2, wherein the angiogenesis inhibitor is bevacizumab, vandetanib, sorafenib, sunitinib or BIBF 1120.

7. The pharmaceutical composition of claim 2, wherein the angiogenesis inhibitor is a compound of formula 1 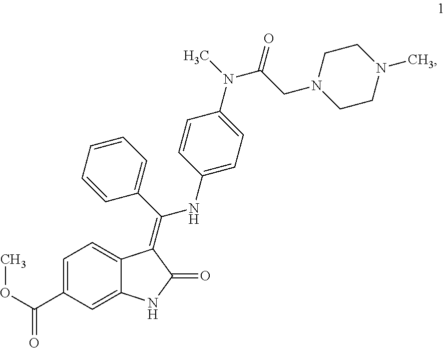 [SMILES: COC(=O)c1ccc2c(c1)NC(=O)/C2=C(\Nc1ccc(N(C)C(=O)CN2CCN(C)CC2)cc1)c1ccccc1]
or a tautomer or pharmaceutically acceptable salt thereof.

8. The pharmaceutical composition of claim 3, wherein the cell signalling inhibitor is a compound targeting epidermal growth factor receptor EGFR.

9. The pharmaceutical composition of claim 3, wherein the cell signalling inhibitor is a compound of formula 3 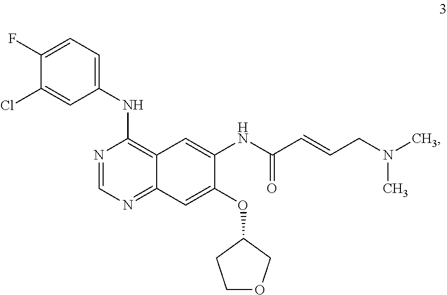 [SMILES: CN(C)C/C=C/C(=O)Nc1cc2c(Nc3ccc(F)c(Cl)c3)ncnc2cc1O[C@H]1CCOC1]
or a tautomer or pharmaceutically acceptable salt thereof.

10. The pharmaceutical composition of claim 1, wherein the Aurora kinase inhibitor (AKI compound) is selected from the group consisting of:

| No. | AKI compound |
|---|---|
| 1 | 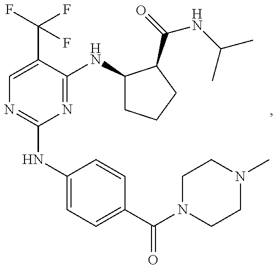 [SMILES: CC(C)NC(=O)[C@H]1CCC[C@H]1Nc1nc(Nc2ccc(C(=O)N3CCN(C)CC3)cc2)ncc1C(F)(F)F] |
| 2 | 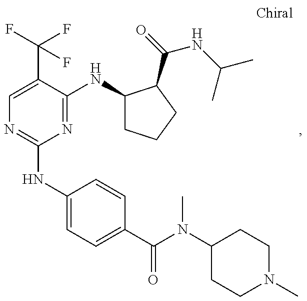 [SMILES: CC(C)NC(=O)[C@H]1CCC[C@H]1Nc1nc(Nc2ccc(C(=O)N(C)C3CCN(C)CC3)cc2)ncc1C(F)(F)F] |
| 3 | 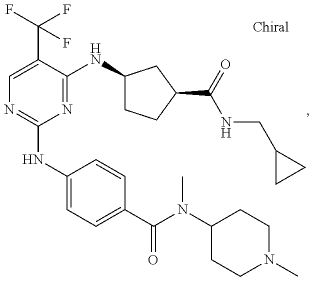 [SMILES: CN1CCC(N(C)C(=O)c2ccc(Nc3ncc(C(F)(F)F)c(N[C@@H]4CC[C@H](C(=O)NCC5CC5)C4)n3)cc2)CC1] |
| 4 | 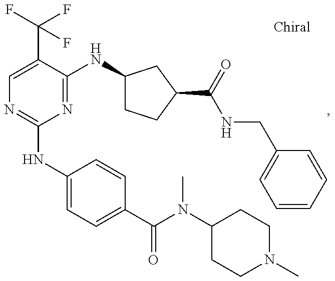 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1] |
| 5 | 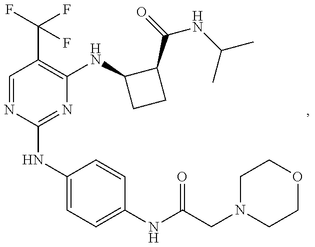 [SMILES: CC(C)NC(=O)[C@H]1CC[C@H]1Nc1nc(Nc2ccc(NC(=O)CN3CCOCC3)cc2)ncc1C(F)(F)F] |
| 6 | 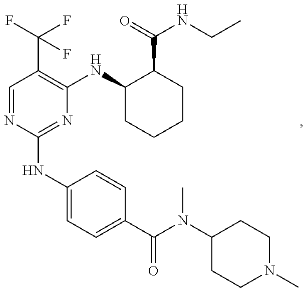 [SMILES: CCNC(=O)[C@H]1CCCC[C@H]1Nc1nc(Nc2ccc(C(=O)N(C)C3CCN(C)CC3)cc2)ncc1C(F)(F)F] |
| 7 | 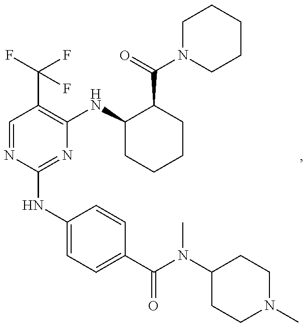 [SMILES: CN1CCC(N(C)C(=O)c2ccc(Nc3ncc(C(F)(F)F)c(N[C@@H]4CCCC[C@@H]4C(=O)N4CCCCC4)n3)cc2)CC1] |
| 8 | 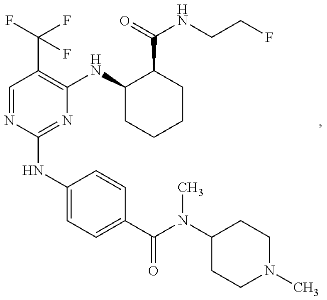 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 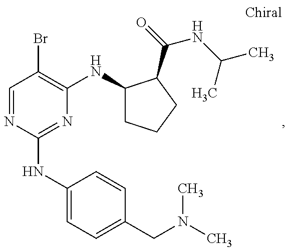 [SMILES: CC(C)NC(=O)[C@H]1CCC[C@H]1Nc1nc(Nc2ccc(CN(C)C)cc2)ncc1Br] |
| 10 | 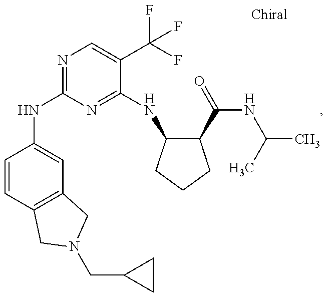 [SMILES: CC(C)NC(=O)[C@H]1CCC[C@H]1Nc1nc(Nc2ccc3c(c2)CN(CC2CC2)C3)ncc1C(F)(F)F] |
| 11 | 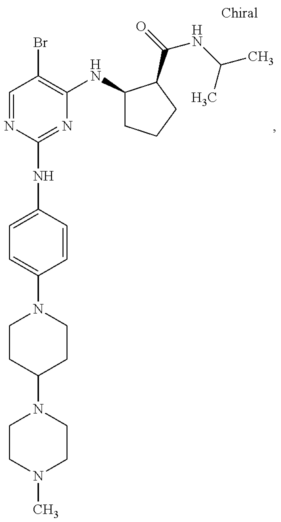 [SMILES: CC(C)NC(=O)[C@H]1CCC[C@H]1Nc1nc(Nc2ccc(N3CCC(N4CCN(C)CC4)CC3)cc2)ncc1Br] |
| 12 | 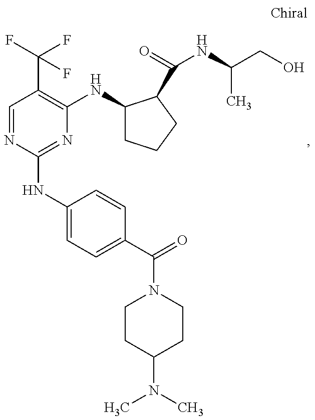 [SMILES: C[C@H](CO)NC(=O)[C@H]1CCC[C@H]1Nc1nc(Nc2ccc(C(=O)N3CCC(N(C)C)CC3)cc2)ncc1C(F)(F)F] |
| 13 | 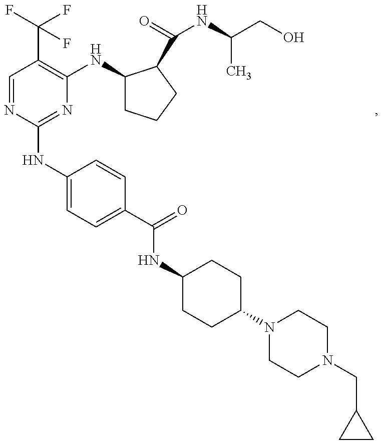 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F] |
| 14 | 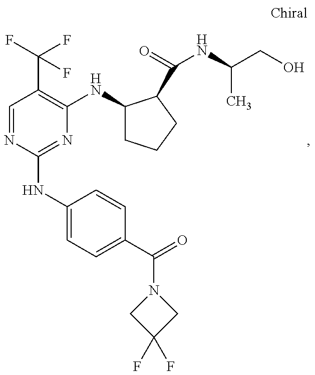 [SMILES: C[C@H](CO)NC(=O)[C@H]1CCC[C@H]1Nc1nc(Nc2ccc(C(=O)N3CC(F)(F)C3)cc2)ncc1C(F)(F)F] |
| 15 | 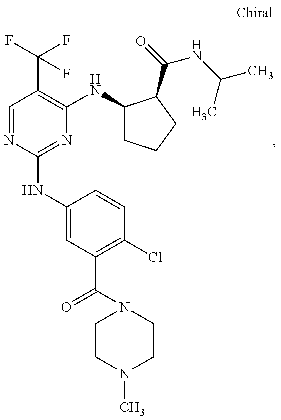 [SMILES: CC(C)NC(=O)[C@H]1CCC[C@H]1Nc1nc(Nc2ccc(Cl)c(C(=O)N3CCN(C)CC3)c2)ncc1C(F)(F)F] |
| 16 | 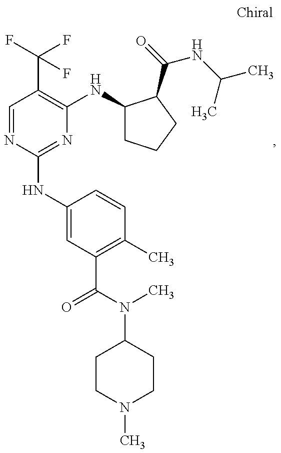 [SMILES: Cc1ccc(Nc2ncc(C(F)(F)F)c(N[C@@H]3CCC[C@@H]3C(=O)NC(C)C)n2)cc1C(=O)N(C)C1CCN(C)CC1] |
| 17 | 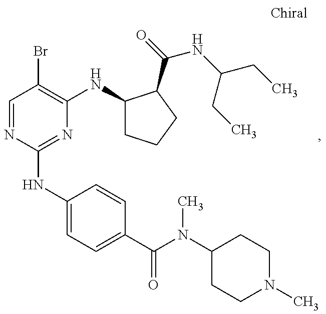 [SMILES: CCC(CC)NC(=O)[C@H]1CCC[C@H]1Nc1nc(Nc2ccc(C(=O)N(C)C3CCN(C)CC3)cc2)ncc1Br] |
| 18 | 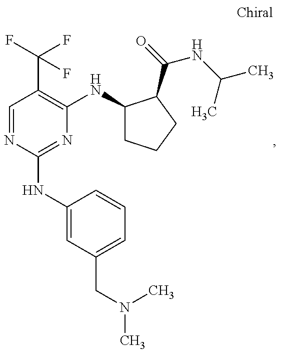 [SMILES: CC(C)NC(=O)[C@H]1CCC[C@H]1Nc1nc(Nc2cccc(CN(C)C)c2)ncc1C(F)(F)F] |
| 19 | 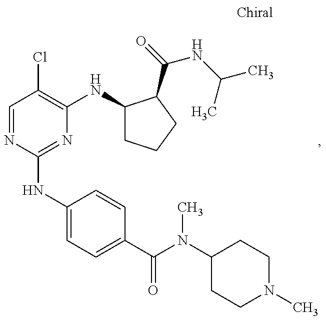 [SMILES: CC(C)NC(=O)[C@H]1CCC[C@H]1Nc1nc(Nc2ccc(C(=O)N(C)C3CCN(C)CC3)cc2)ncc1Cl] |
| 20 | 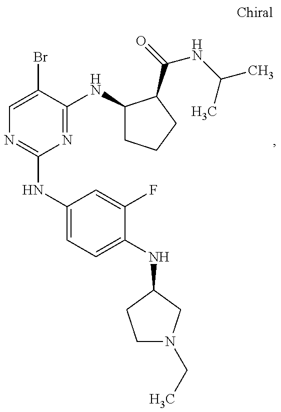 [SMILES: CCN1CC[C@@H](Nc2ccc(Nc3ncc(Br)c(N[C@@H]4CCC[C@@H]4C(=O)NC(C)C)n3)cc2F)C1] |
| 21 | 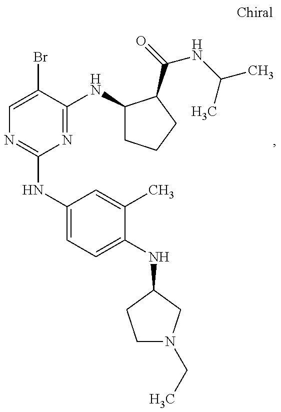 [SMILES: CCN1CC[C@@H](Nc2ccc(Nc3ncc(Br)c(N[C@@H]4CCC[C@@H]4C(=O)NC(C)C)n3)cc2C)C1] |
| 22 | 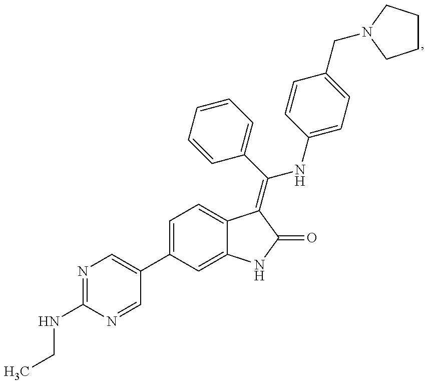 [SMILES: CCNc1ncc(-c2ccc3c(c2)NC(=O)/C3=C(\Nc2ccc(CN3CCCC3)cc2)c2ccccc2)cn1] |
| 23 | 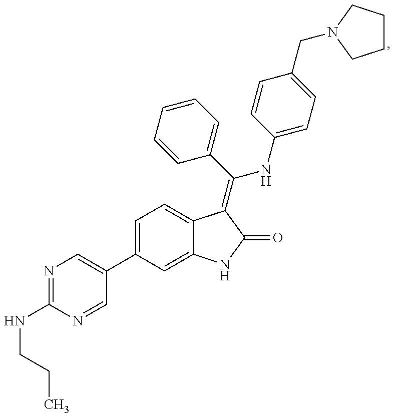 [SMILES: CCCNc1ncc(-c2ccc3c(c2)NC(=O)/C3=C(\Nc2ccc(CN3CCCC3)cc2)c2ccccc2)cn1] |
| 24 | 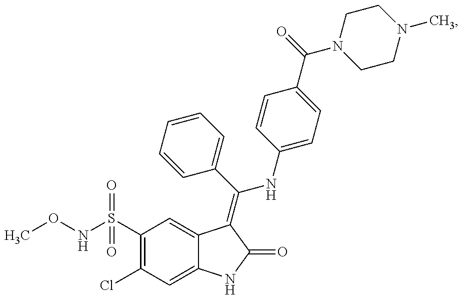 [SMILES: CONS(=O)(=O)c1cc2c(cc1Cl)NC(=O)/C2=C(\Nc1ccc(C(=O)N2CCN(C)CC2)cc1)c1ccccc1] |
| 25 | 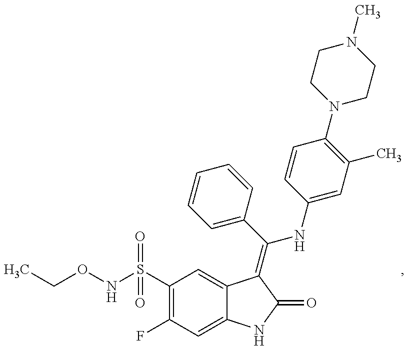 [SMILES: CCONS(=O)(=O)c1cc2c(cc1F)NC(=O)/C2=C(\Nc1ccc(N2CCN(C)CC2)c(C)c1)c1ccccc1] |
| 26 | 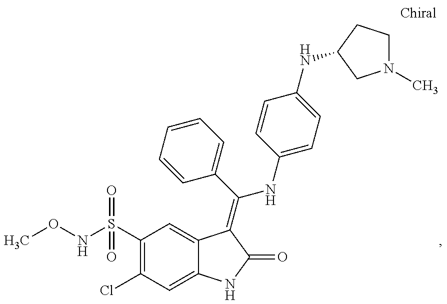 |
| 27 | 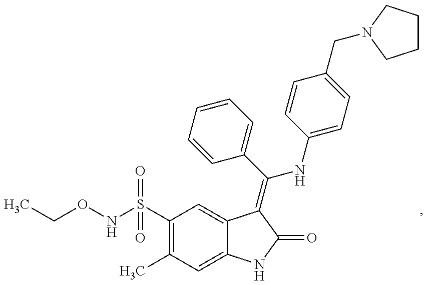 [SMILES: CCONS(=O)(=O)c1cc2c(cc1C)NC(=O)/C2=C(\Nc1ccc(CN2CCCC2)cc1)c1ccccc1] |
| 28 | 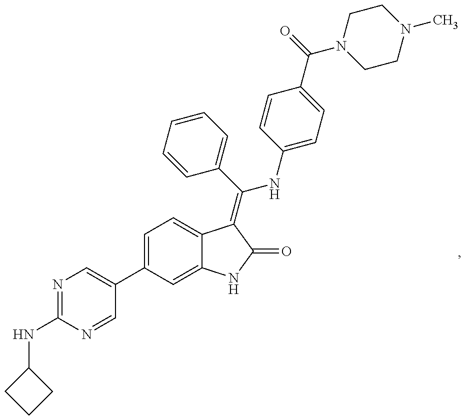 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 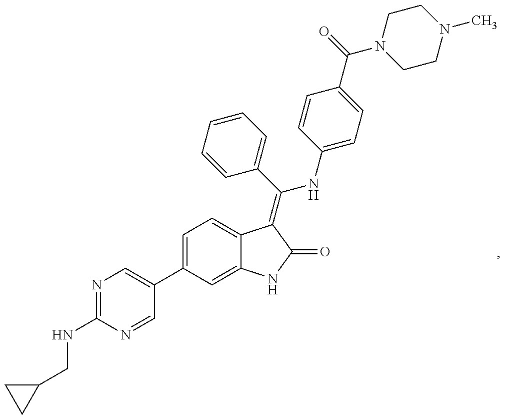 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 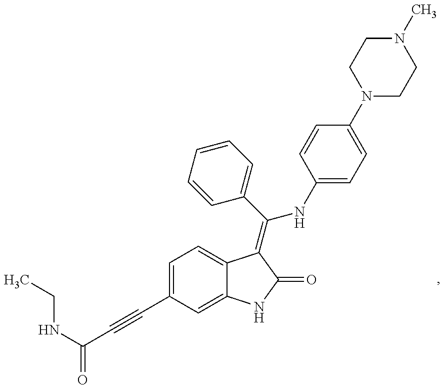 [SMILES: CCNC(=O)C#Cc1ccc2c(c1)NC(=O)/C2=C(\Nc1ccc(N2CCN(C)CC2)cc1)c1ccccc1] |
| 31 | 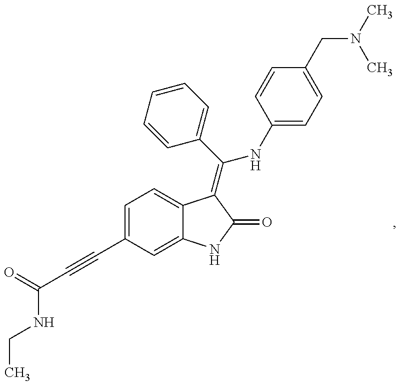 [SMILES: CCNC(=O)C#Cc1ccc2c(c1)NC(=O)/C2=C(\Nc1ccc(CN(C)C)cc1)c1ccccc1] |
| 32 | 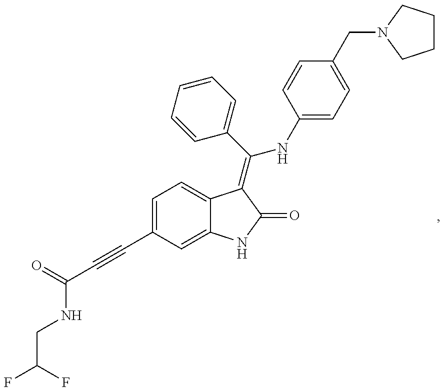 [SMILES: O=C(C#Cc1ccc2c(c1)NC(=O)/C2=C(\Nc1ccc(CN2CCCC2)cc1)c1ccccc1)NCC(F)F] |
| 33 | 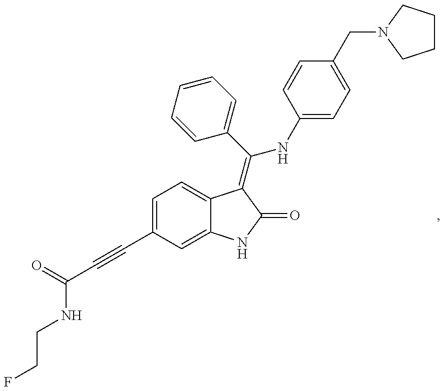 [SMILES: O=C(C#Cc1ccc2c(c1)NC(=O)/C2=C(\Nc1ccc(CN2CCCC2)cc1)c1ccccc1)NCCF] |
| 34 | 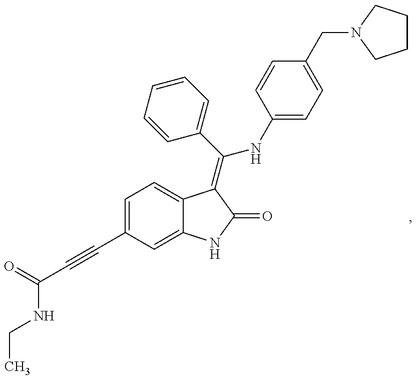 [SMILES: CCNC(=O)C#Cc1ccc2c(c1)NC(=O)/C2=C(\Nc1ccc(CN2CCCC2)cc1)c1ccccc1] |
| 35 | 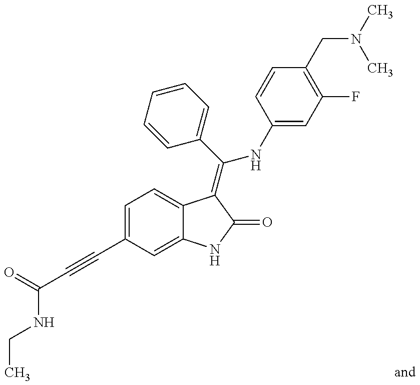 [SMILES: CCNC(=O)C#Cc1ccc2c(c1)NC(=O)/C2=C(\Nc1ccc(CN(C)C)c(F)c1)c1ccccc1] |
| 36 | 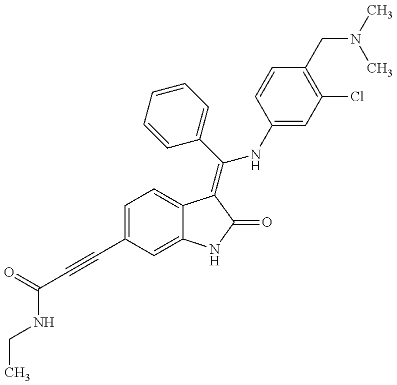 [SMILES: CCNC(=O)C#Cc1ccc2c(c1)NC(=O)/C2=C(\Nc1ccc(CN(C)C)c(Cl)c1)c1ccccc1] |

or a tautomer or pharmaceutically acceptable salt thereof.

11. The pharmaceutical composition of claim 10 wherein the wherein the cell signalling and/or angiogenesis inhibitor is an angiogenesis inhibitor which is a compound of formula 1 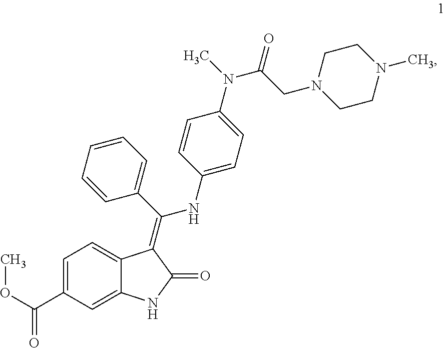 [SMILES: COC(=O)c1ccc2c(c1)NC(=O)/C2=C(\Nc1ccc(N(C)C(=O)CN2CCN(C)CC2)cc1)c1ccccc1]
or a tautomer or pharmaceutically acceptable salt thereof.

12. The pharmaceutical composition of claim 11, wherein the angiogeneis inhibitor is the hydroethanesulphonate (1a)

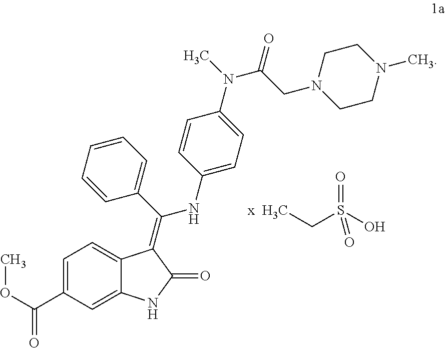

13. The pharmaceutical composition of claim 10, wherein the cell signalling and/or angiogenesis inhibitor is a cell signalling inhibitor which is a compound of formula 3 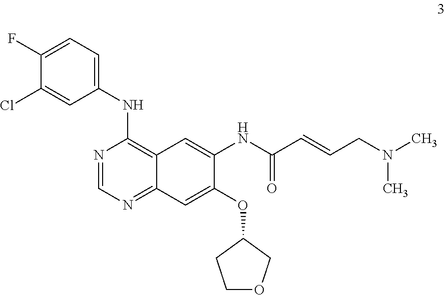 [SMILES: CN(C)C/C=C/C(=O)Nc1cc2c(Nc3ccc(F)c(Cl)c3)ncnc2cc1O[C@H]1CCOC1]
or a tautomer or pharmaceutically acceptable salt thereof.

14. A pharmaceutical composition of claim 2, further comprising a compound of formula 3 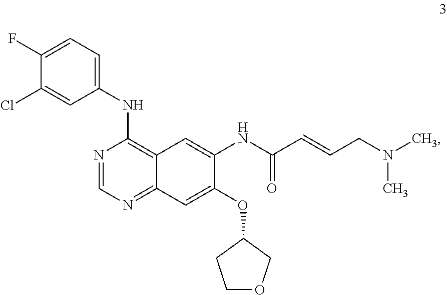 [SMILES: CN(C)C/C=C/C(=O)Nc1cc2c(Nc3ccc(F)c(Cl)c3)ncnc2cc1O[C@H]1CCOC1]
or a tautomer or pharmaceutically acceptable salt thereof.

15. A kit including a first pharmaceutical composition which comprises a compound of formula 1 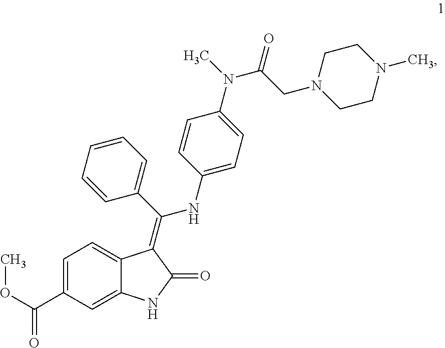 [SMILES: COC(=O)c1ccc2c(c1)NC(=O)/C2=C(\Nc1ccc(N(C)C(=O)CN2CCN(C)CC2)cc1)c1ccccc1]
or a tautomer or pharmaceutically acceptable salt thereof, and a second pharmaceutical composition which comprises a compound selected from the group consisting of:

| No. | AKI compound |
|---|---|
| 1 | 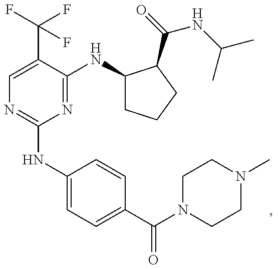 [SMILES: CC(C)NC(=O)[C@H]1CCC[C@H]1Nc1nc(Nc2ccc(C(=O)N3CCN(C)CC3)cc2)ncc1C(F)(F)F] |
| 2 | 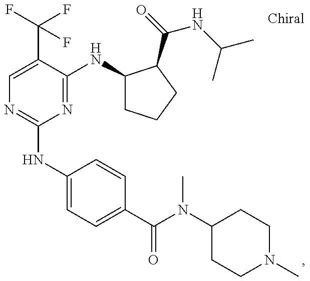 [SMILES: CC(C)NC(=O)[C@H]1CCC[C@H]1Nc1nc(Nc2ccc(C(=O)N(C)C3CCN(C)CC3)cc2)ncc1C(F)(F)F] |
| 3 | 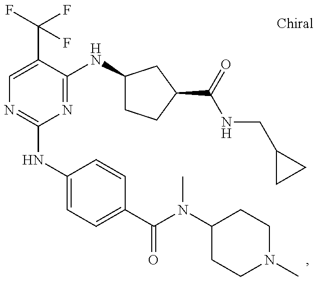 [SMILES: CN1CCC(N(C)C(=O)c2ccc(Nc3ncc(C(F)(F)F)c(N[C@@H]4CC[C@H](C(=O)NCC5CC5)C4)n3)cc2)CC1] |
| 4 | 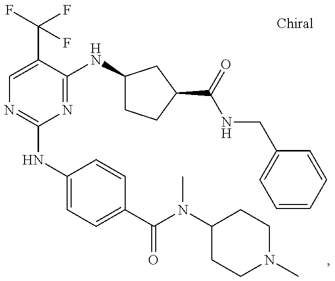 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1] |
| 5 | 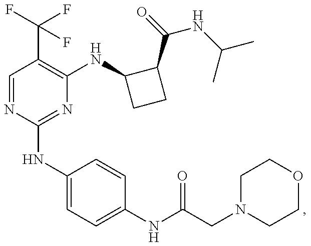 [SMILES: CC(C)NC(=O)[C@H]1CC[C@H]1Nc1nc(Nc2ccc(NC(=O)CN3CCOCC3)cc2)ncc1C(F)(F)F] |
| 6 | 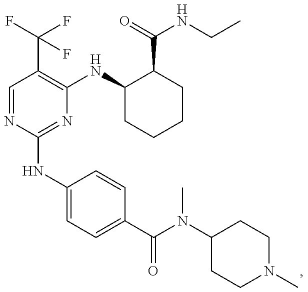 [SMILES: CCNC(=O)[C@H]1CCCC[C@H]1Nc1nc(Nc2ccc(C(=O)N(C)C3CCN(C)CC3)cc2)ncc1C(F)(F)F] |
| 7 | 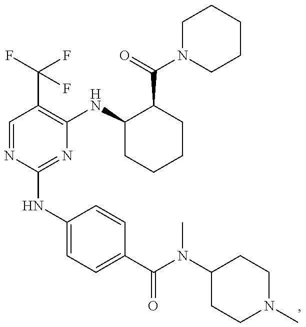 [SMILES: CN1CCC(N(C)C(=O)c2ccc(Nc3ncc(C(F)(F)F)c(N[C@@H]4CCCC[C@@H]4C(=O)N4CCCCC4)n3)cc2)CC1] |
| 8 | 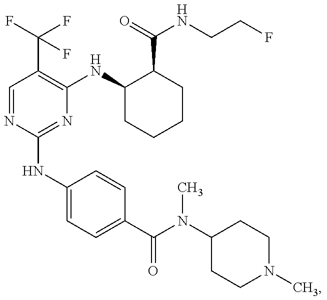 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 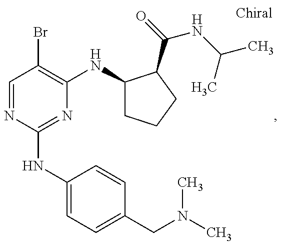 [SMILES: CC(C)NC(=O)[C@H]1CCC[C@H]1Nc1nc(Nc2ccc(CN(C)C)cc2)ncc1Br] |
| 10 | 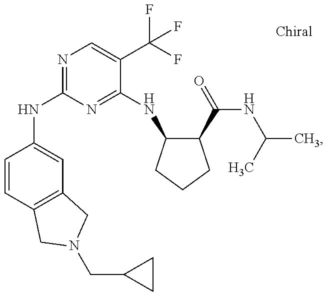 [SMILES: CC(C)NC(=O)[C@H]1CCC[C@H]1Nc1nc(Nc2ccc3c(c2)CN(CC2CC2)C3)ncc1C(F)(F)F] |
| 11 | 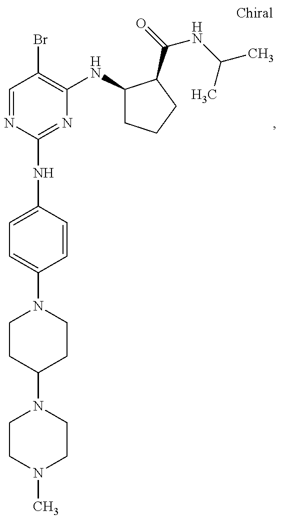 [SMILES: CC(C)NC(=O)[C@H]1CCC[C@H]1Nc1nc(Nc2ccc(N3CCC(N4CCN(C)CC4)CC3)cc2)ncc1Br] |
| 12 | 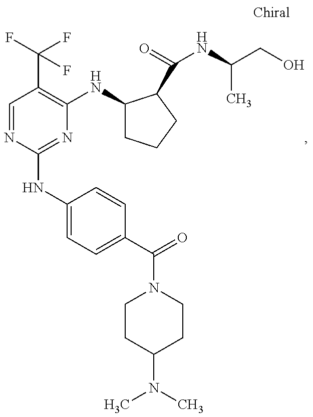 [SMILES: C[C@H](CO)NC(=O)[C@H]1CCC[C@H]1Nc1nc(Nc2ccc(C(=O)N3CCC(N(C)C)CC3)cc2)ncc1C(F)(F)F] |
| 13 | 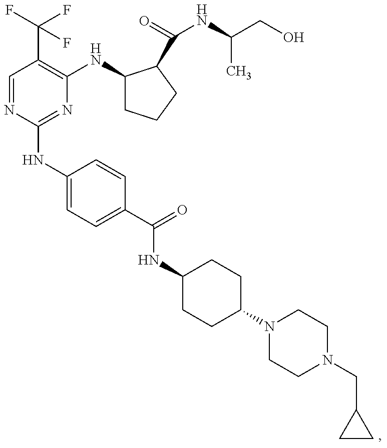 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F] |
| 14 | 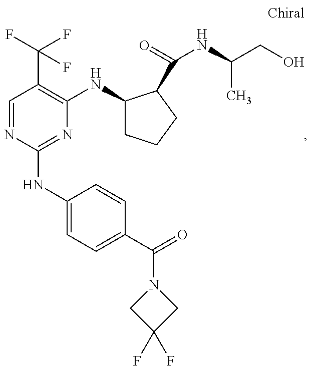 [SMILES: C[C@H](CO)NC(=O)[C@H]1CCC[C@H]1Nc1nc(Nc2ccc(C(=O)N3CC(F)(F)C3)cc2)ncc1C(F)(F)F] |
| 15 | 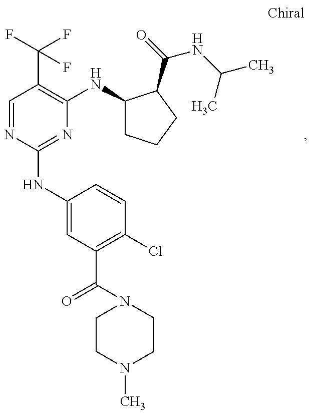 [SMILES: CC(C)NC(=O)[C@H]1CCC[C@H]1Nc1nc(Nc2ccc(Cl)c(C(=O)N3CCN(C)CC3)c2)ncc1C(F)(F)F] |
| 16 | 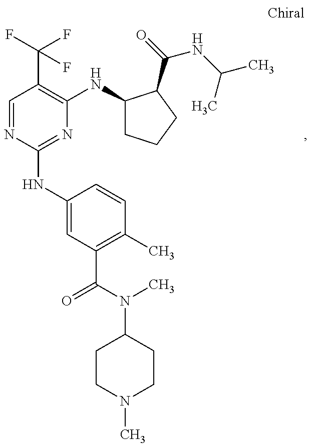 [SMILES: Cc1ccc(Nc2ncc(C(F)(F)F)c(N[C@@H]3CCC[C@@H]3C(=O)NC(C)C)n2)cc1C(=O)N(C)C1CCN(C)CC1] |
| 17 | 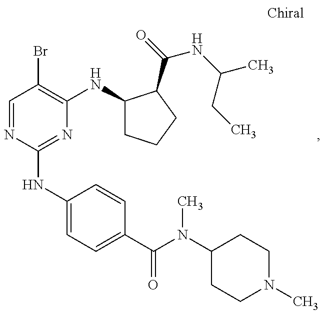 [SMILES: CCC(C)NC(=O)[C@H]1CCC[C@H]1Nc1nc(Nc2ccc(C(=O)N(C)C3CCN(C)CC3)cc2)ncc1Br] |
| 18 | 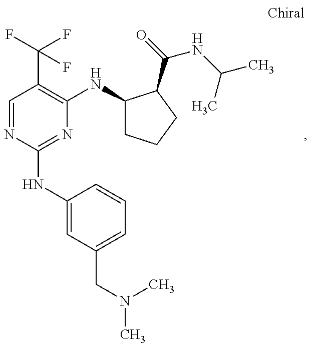 [SMILES: CC(C)NC(=O)[C@H]1CCC[C@H]1Nc1nc(Nc2cccc(CN(C)C)c2)ncc1C(F)(F)F] |
| 19 | 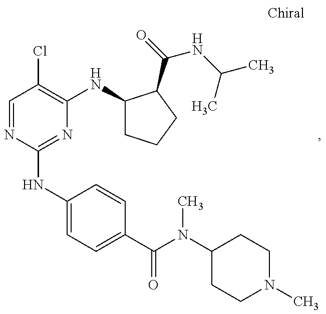 [SMILES: CC(C)NC(=O)[C@H]1CCC[C@H]1Nc1nc(Nc2ccc(C(=O)N(C)C3CCN(C)CC3)cc2)ncc1Cl] |
| 20 | 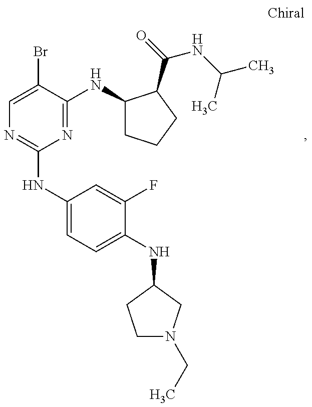 [SMILES: CCN1CC[C@@H](Nc2ccc(Nc3ncc(Br)c(N[C@@H]4CCC[C@@H]4C(=O)NC(C)C)n3)cc2F)C1] |
| 21 | 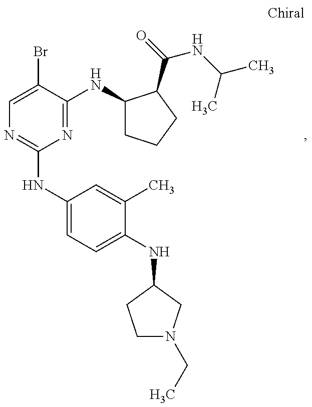 [SMILES: CCN1CC[C@@H](Nc2ccc(Nc3ncc(Br)c(N[C@@H]4CCC[C@@H]4C(=O)NC(C)C)n3)cc2C)C1] |
| 22 | 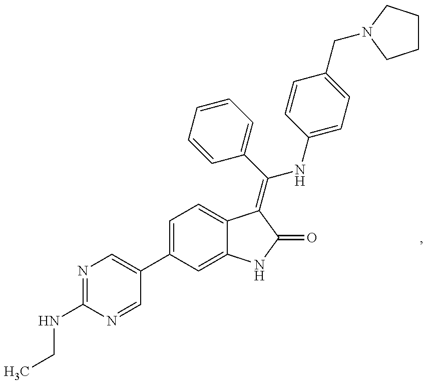 [SMILES: CCNc1ncc(-c2ccc3c(c2)NC(=O)/C3=C(\Nc2ccc(CN3CCCC3)cc2)c2ccccc2)cn1] |
| 23 | 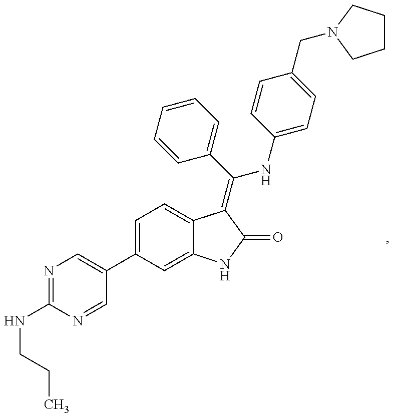 [SMILES: CCCNc1ncc(-c2ccc3c(c2)NC(=O)/C3=C(\Nc2ccc(CN3CCCC3)cc2)c2ccccc2)cn1] |
| 24 | 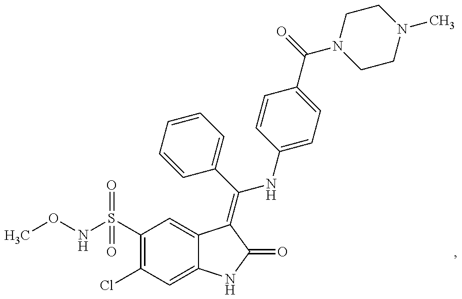 |
| 25 | 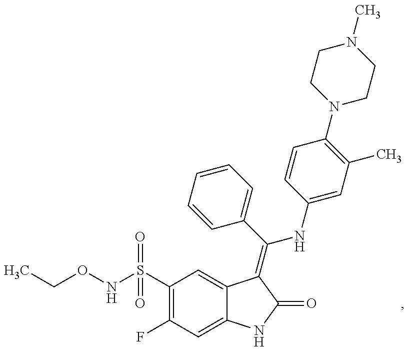 [SMILES: CCONS(=O)(=O)c1cc2c(cc1F)NC(=O)/C2=C(\Nc1ccc(N2CCN(C)CC2)c(C)c1)c1ccccc1] |
| 26 | 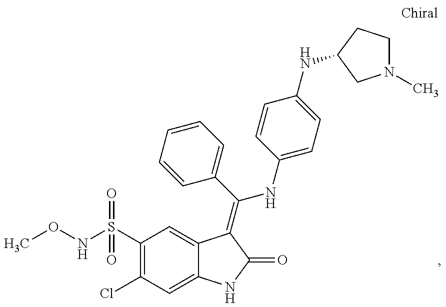 |
| 27 | 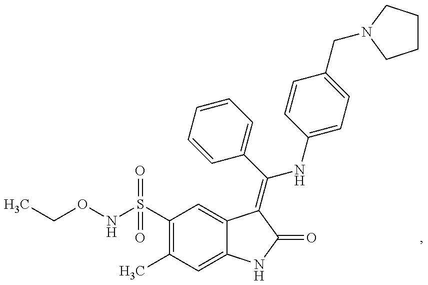 [SMILES: CCONS(=O)(=O)c1cc2c(cc1C)NC(=O)/C2=C(\Nc1ccc(CN2CCCC2)cc1)c1ccccc1] |
| 28 | 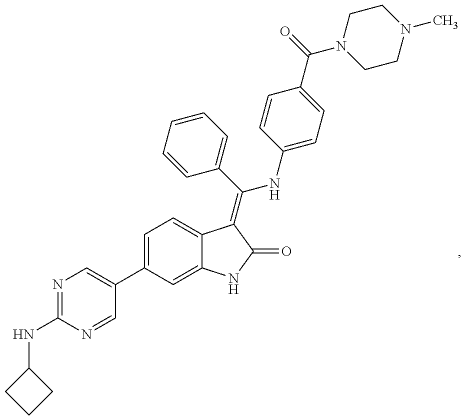 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 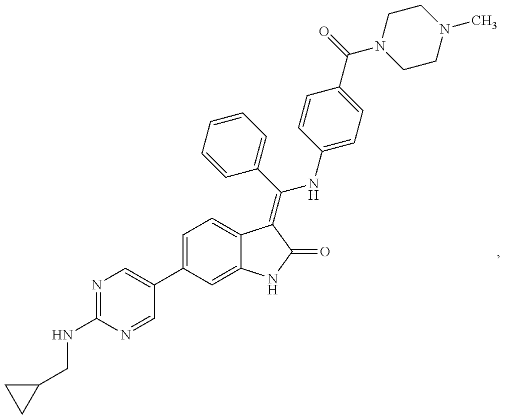 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 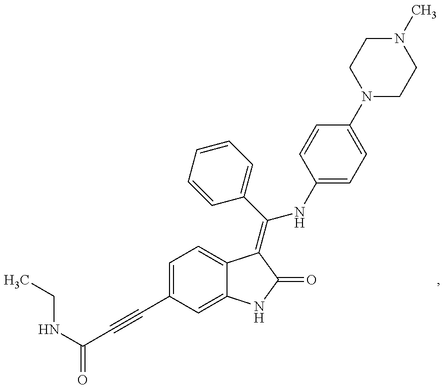 [SMILES: CCNC(=O)C#Cc1ccc2c(c1)NC(=O)/C2=C(\Nc1ccc(N2CCN(C)CC2)cc1)c1ccccc1] |
| 31 | 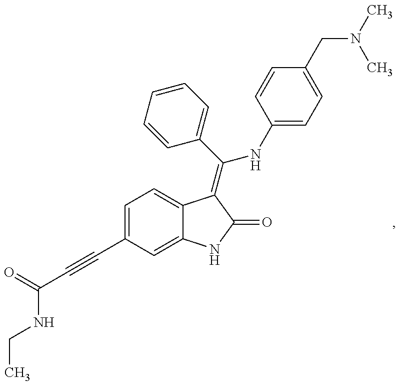 [SMILES: CCNC(=O)C#Cc1ccc2c(c1)NC(=O)/C2=C(\Nc1ccc(CN(C)C)cc1)c1ccccc1] |
| 32 | 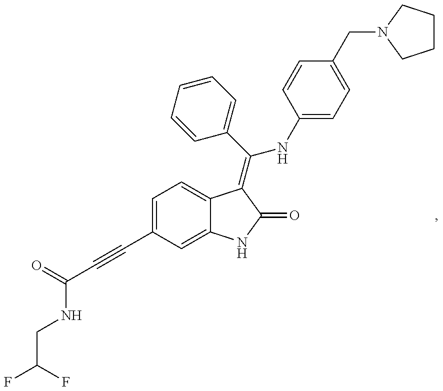 [SMILES: O=C(C#Cc1ccc2c(c1)NC(=O)/C2=C(\Nc1ccc(CN2CCCC2)cc1)c1ccccc1)NCC(F)F] |
| 33 | 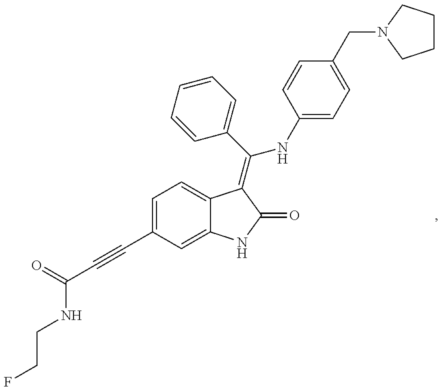 [SMILES: O=C(C#Cc1ccc2c(c1)NC(=O)/C2=C(\Nc1ccc(CN2CCCC2)cc1)c1ccccc1)NCCF] |
| 34 | 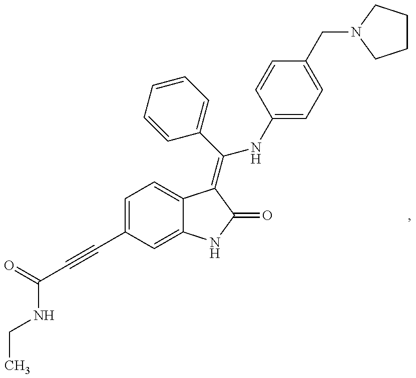 [SMILES: CCNC(=O)C#Cc1ccc2c(c1)NC(=O)/C2=C(\Nc1ccc(CN2CCCC2)cc1)c1ccccc1] |
| 35 | 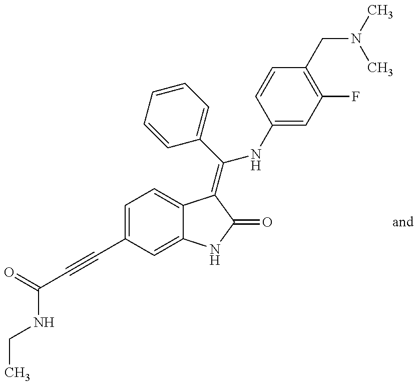 [SMILES: CCNC(=O)C#Cc1ccc2c(c1)NC(=O)/C2=C(\Nc1ccc(CN(C)C)c(F)c1)c1ccccc1] |
| 36 | 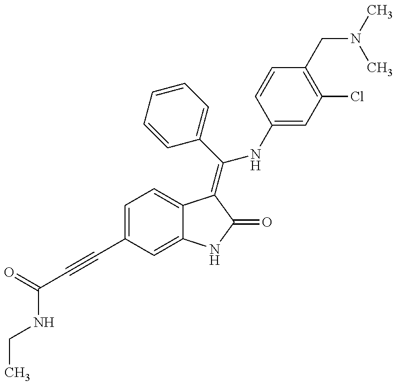 [SMILES: CCNC(=O)C#Cc1ccc2c(c1)NC(=O)/C2=C(\Nc1ccc(CN(C)C)c(Cl)c1)c1ccccc1] |

or a tautomer or pharmaceutically acceptable salt thereof.

16. A kit including a first pharmaceutical composition which comprises a compound of formula 3 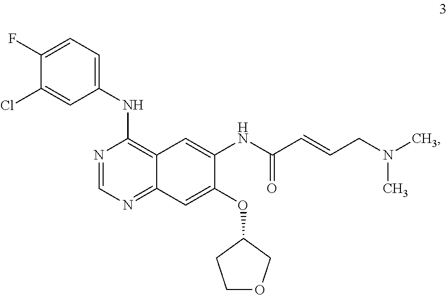 [SMILES: CN(C)C/C=C/C(=O)Nc1cc2c(Nc3ccc(F)c(Cl)c3)ncnc2cc1O[C@H]1CCOC1]
or a tautomer or pharmaceutically acceptable salt thereof, and a second pharmaceutical composition which comprises a compound selected from the group consisting of:

| No. | AKI compound |
|---|---|
| 1 | 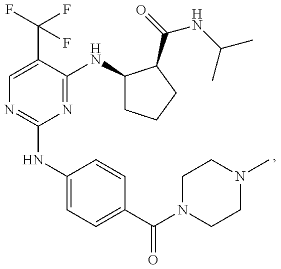 [SMILES: CC(C)NC(=O)[C@H]1CCC[C@H]1Nc1nc(Nc2ccc(C(=O)N3CCN(C)CC3)cc2)ncc1C(F)(F)F] |
| 2 | 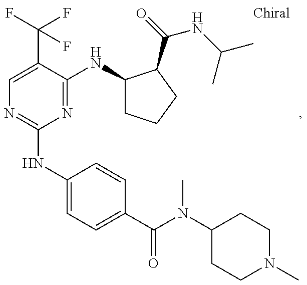 [SMILES: CC(C)NC(=O)[C@H]1CCC[C@H]1Nc1nc(Nc2ccc(C(=O)N(C)C3CCN(C)CC3)cc2)ncc1C(F)(F)F] |
| 3 | 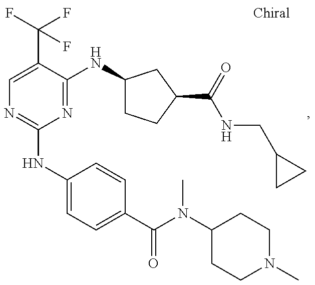 [SMILES: CN1CCC(N(C)C(=O)c2ccc(Nc3ncc(C(F)(F)F)c(N[C@@H]4CC[C@H](C(=O)NCC5CC5)C4)n3)cc2)CC1] |
| 4 | 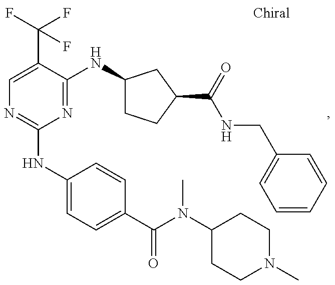 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1] |
| 5 | 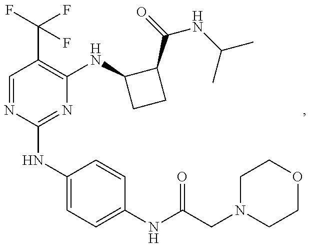 [SMILES: CC(C)NC(=O)[C@H]1CC[C@H]1Nc1nc(Nc2ccc(NC(=O)CN3CCOCC3)cc2)ncc1C(F)(F)F] |
| 6 | 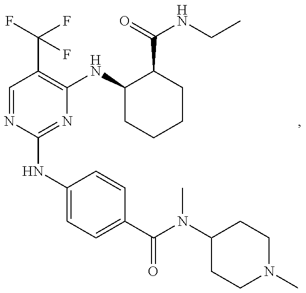 [SMILES: CCNC(=O)[C@H]1CCCC[C@H]1Nc1nc(Nc2ccc(C(=O)N(C)C3CCN(C)CC3)cc2)ncc1C(F)(F)F] |
| 7 | 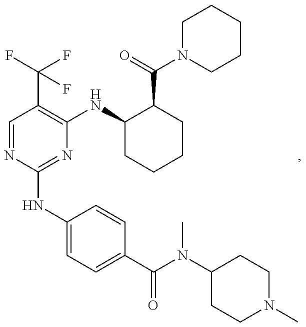 [SMILES: CN1CCC(N(C)C(=O)c2ccc(Nc3ncc(C(F)(F)F)c(N[C@@H]4CCCC[C@@H]4C(=O)N4CCCCC4)n3)cc2)CC1] |
| 8 | 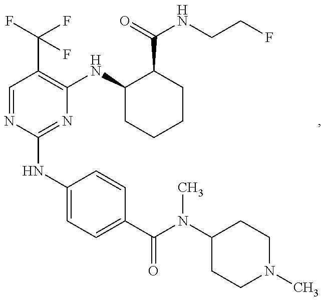 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 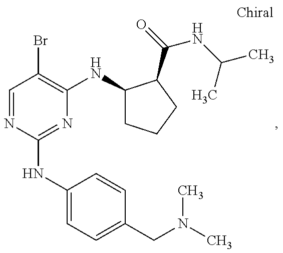 [SMILES: CC(C)NC(=O)[C@H]1CCC[C@H]1Nc1nc(Nc2ccc(CN(C)C)cc2)ncc1Br] |
| 10 | 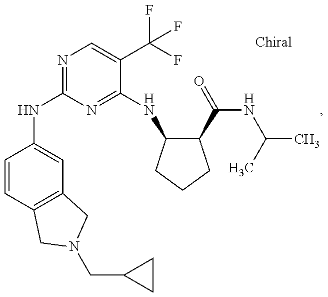 [SMILES: CC(C)NC(=O)[C@H]1CCC[C@H]1Nc1nc(Nc2ccc3c(c2)CN(CC2CC2)C3)ncc1C(F)(F)F] |
| 11 | 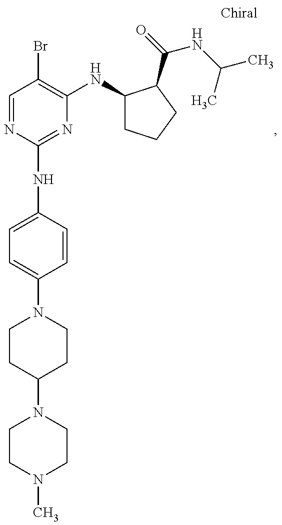 [SMILES: CC(C)NC(=O)[C@H]1CCC[C@H]1Nc1nc(Nc2ccc(N3CCC(N4CCN(C)CC4)CC3)cc2)ncc1Br] |
| 12 | 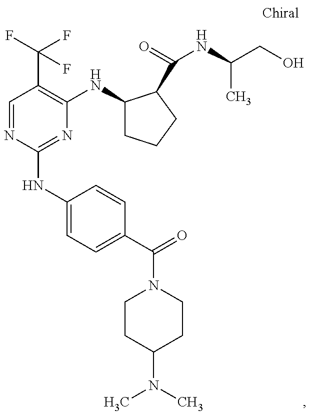 [SMILES: C[C@H](CO)NC(=O)[C@H]1CCC[C@H]1Nc1nc(Nc2ccc(C(=O)N3CCC(N(C)C)CC3)cc2)ncc1C(F)(F)F] |
| 13 | 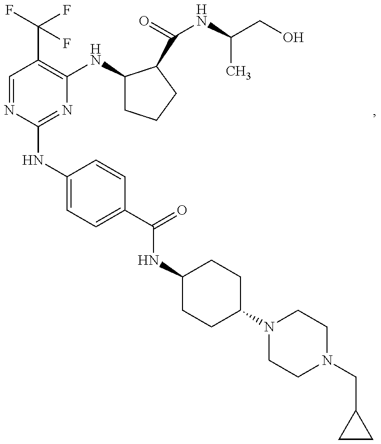 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F] |
| 14 | 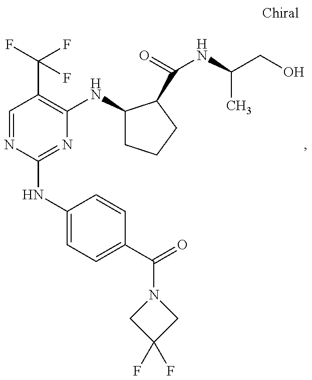 [SMILES: C[C@H](CO)NC(=O)[C@H]1CCC[C@H]1Nc1nc(Nc2ccc(C(=O)N3CC(F)(F)C3)cc2)ncc1C(F)(F)F] |
| 15 | 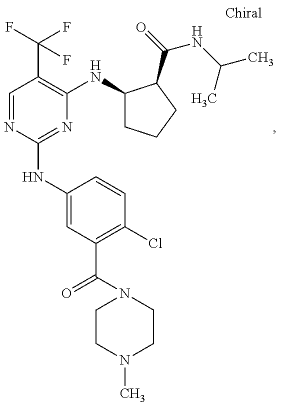 [SMILES: CC(C)NC(=O)[C@H]1CCC[C@H]1Nc1nc(Nc2ccc(Cl)c(C(=O)N3CCN(C)CC3)c2)ncc1C(F)(F)F] |
| 16 | 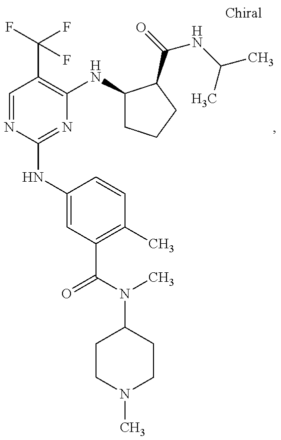 [SMILES: Cc1ccc(Nc2ncc(C(F)(F)F)c(N[C@@H]3CCC[C@@H]3C(=O)NC(C)C)n2)cc1C(=O)N(C)C1CCN(C)CC1] |
| 17 | 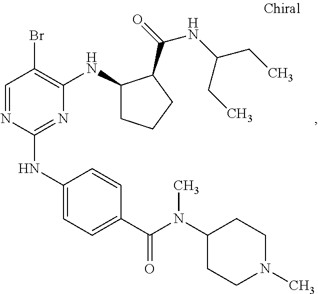 [SMILES: CCC(CC)NC(=O)[C@H]1CCC[C@H]1Nc1nc(Nc2ccc(C(=O)N(C)C3CCN(C)CC3)cc2)ncc1Br] |
| 18 | 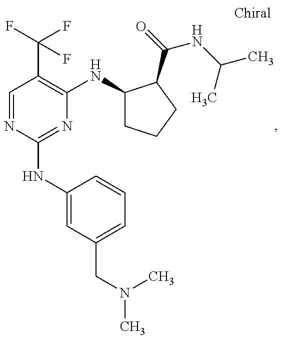 [SMILES: CC(C)NC(=O)[C@H]1CCC[C@H]1Nc1nc(Nc2cccc(CN(C)C)c2)ncc1C(F)(F)F] |
| 19 | 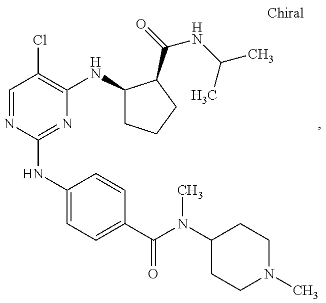 [SMILES: CC(C)NC(=O)[C@H]1CCC[C@H]1Nc1nc(Nc2ccc(C(=O)N(C)C3CCN(C)CC3)cc2)ncc1Cl] |
| 20 | 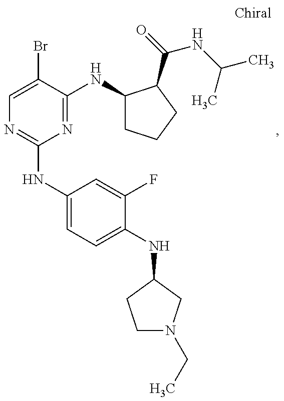 [SMILES: CCN1CC[C@@H](Nc2ccc(Nc3ncc(Br)c(N[C@@H]4CCC[C@@H]4C(=O)NC(C)C)n3)cc2F)C1] |
| 21 | 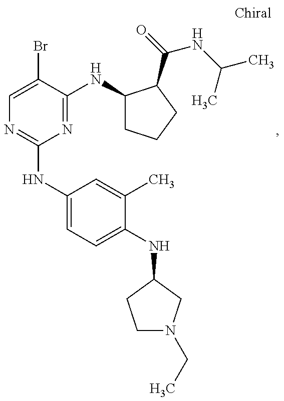 [SMILES: CCN1CC[C@@H](Nc2ccc(Nc3ncc(Br)c(N[C@@H]4CCC[C@@H]4C(=O)NC(C)C)n3)cc2C)C1] |
| 22 | 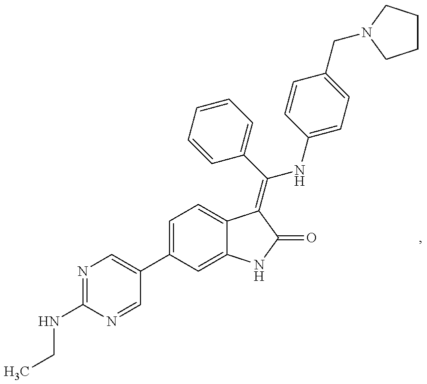 [SMILES: CCNc1ncc(-c2ccc3c(c2)NC(=O)/C3=C(\Nc2ccc(CN3CCCC3)cc2)c2ccccc2)cn1] |
| 23 | 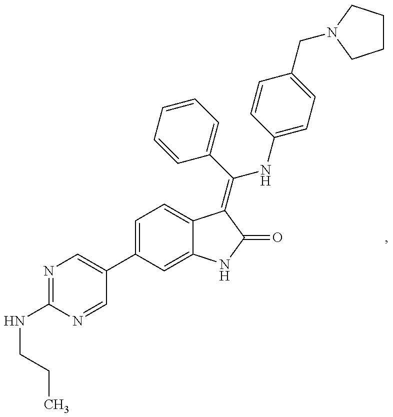 [SMILES: CCCNc1ncc(-c2ccc3c(c2)NC(=O)/C3=C(\Nc2ccc(CN3CCCC3)cc2)c2ccccc2)cn1] |
| 24 | 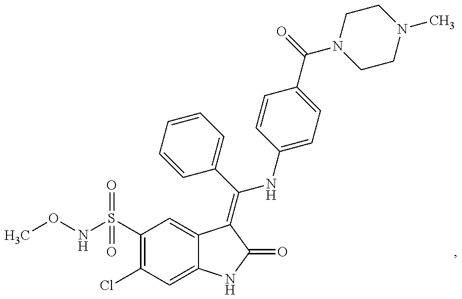 |
| 25 | 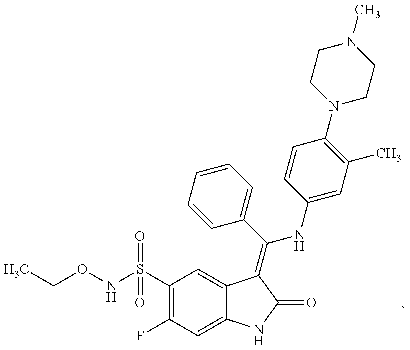 [SMILES: CCONS(=O)(=O)c1cc2c(cc1F)NC(=O)/C2=C(\Nc1ccc(N2CCN(C)CC2)c(C)c1)c1ccccc1] |
| 26 | 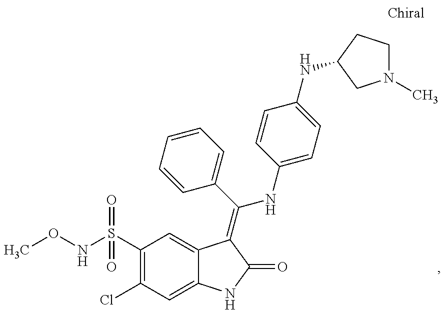 |
| 27 | 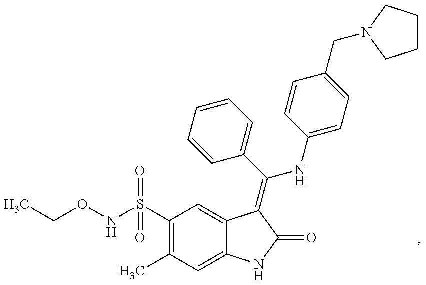 [SMILES: CCONS(=O)(=O)c1cc2c(cc1C)NC(=O)/C2=C(\Nc1ccc(CN2CCCC2)cc1)c1ccccc1] |
| 28 | 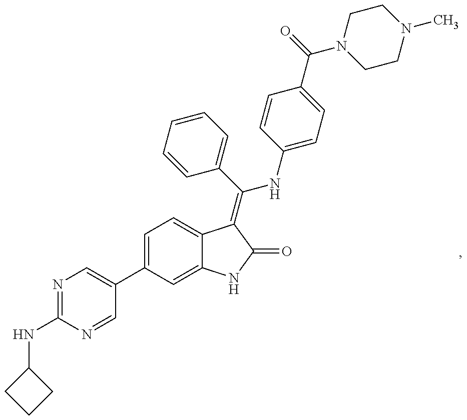 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 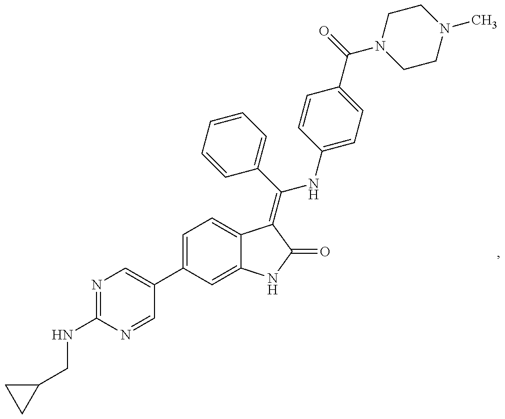 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 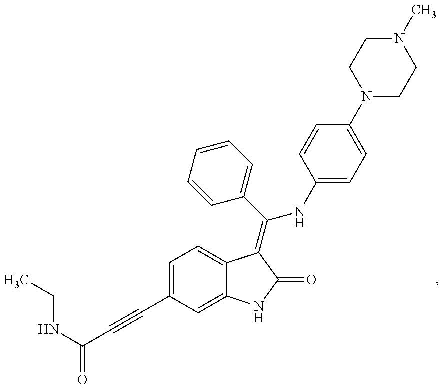 [SMILES: CCNC(=O)C#Cc1ccc2c(c1)NC(=O)/C2=C(\Nc1ccc(N2CCN(C)CC2)cc1)c1ccccc1] |
| 31 | 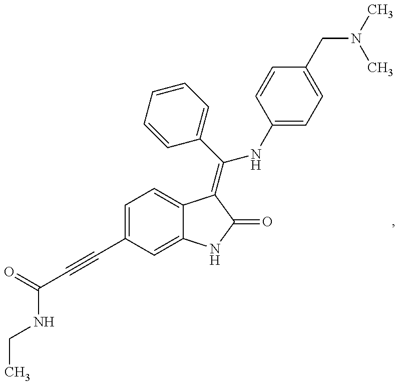 [SMILES: CCNC(=O)C#Cc1ccc2c(c1)NC(=O)/C2=C(\Nc1ccc(CN(C)C)cc1)c1ccccc1] |
| 32 | 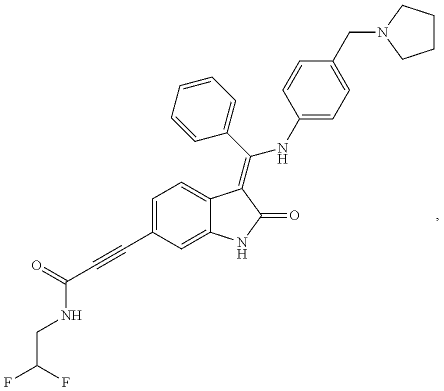 [SMILES: O=C(C#Cc1ccc2c(c1)NC(=O)/C2=C(\Nc1ccc(CN2CCCC2)cc1)c1ccccc1)NCC(F)F] |
| 33 | 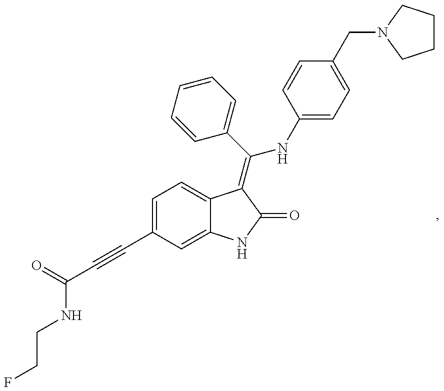 [SMILES: O=C(C#Cc1ccc2c(c1)NC(=O)/C2=C(\Nc1ccc(CN2CCCC2)cc1)c1ccccc1)NCCF] |
| 34 | 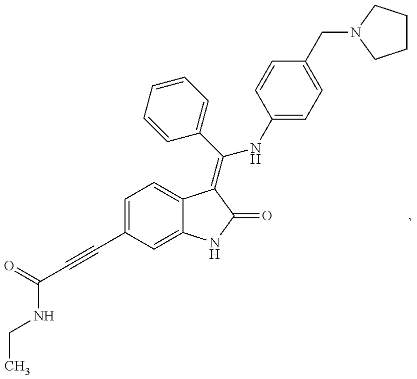 [SMILES: CCNC(=O)C#Cc1ccc2c(c1)NC(=O)/C2=C(\Nc1ccc(CN2CCCC2)cc1)c1ccccc1] |
| 35 | 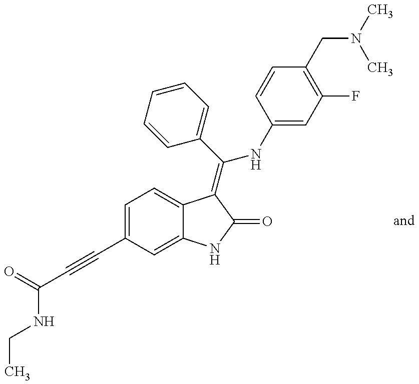 [SMILES: CCNC(=O)C#Cc1ccc2c(c1)NC(=O)/C2=C(\Nc1ccc(CN(C)C)c(F)c1)c1ccccc1] |
| 36 | 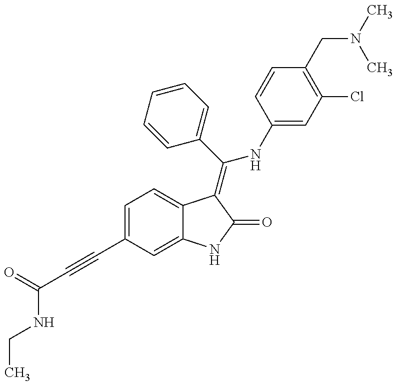 [SMILES: CCNC(=O)C#Cc1ccc2c(c1)NC(=O)/C2=C(\Nc1ccc(CN(C)C)c(Cl)c1)c1ccccc1] |

or a tautomer or pharmaceutically acceptable salt thereof.

17. A method for treating oncological and fibrotic diseases which comprises administering to a host suffering from such disease therapeutically effective amounts of a cell signalling and/or angiogenesis inhibitor and an Aurora kinase inhibitor.

18. The method of claim 17 wherein the cell signalling and/or angiogenesis inhibitor is an angiogenesis inhibitor which is a compound of formula 1 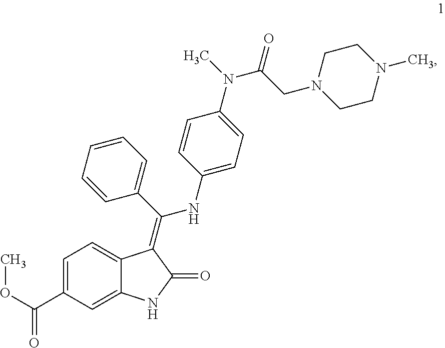 [SMILES: COC(=O)c1ccc2c(c1)NC(=O)/C2=C(\Nc1ccc(N(C)C(=O)CN2CCN(C)CC2)cc1)c1ccccc1]
or a tautomer or pharmaceutically acceptable salt thereof, and the Aurora kinase inhibitor is a compound selected from the group consisting of:

| No. | AKI compound |
|---|---|
| 1 | 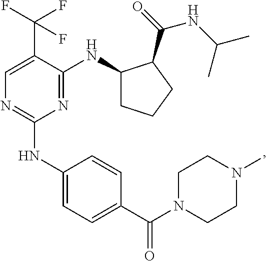 |
| 2 | 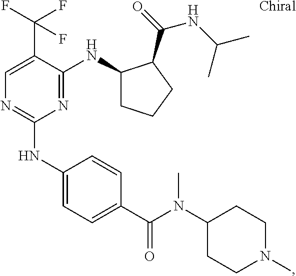 |
| 3 | 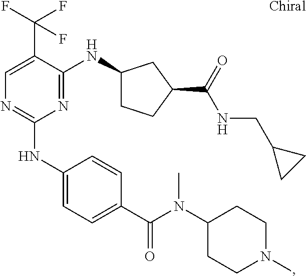 |
| 4 | 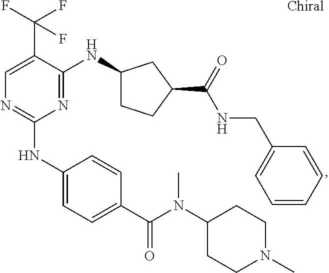 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1 ; "Chiral" ; ","] |
| 5 | 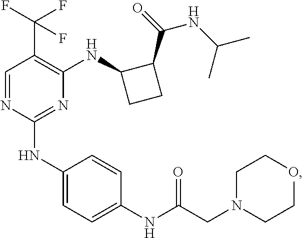 [SMILES: CC(C)NC(=O)[C@H]1CC[C@H]1Nc1nc(Nc2ccc(NC(=O)CN3CCOCC3)cc2)ncc1C(F)(F)F] |
| 6 | 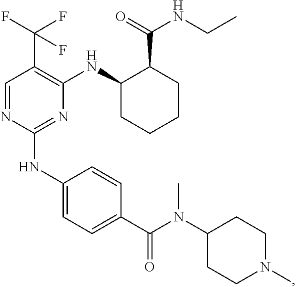 |
| 7 | 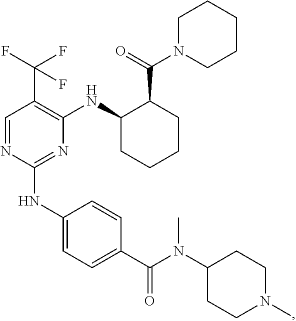 |
| 8 | 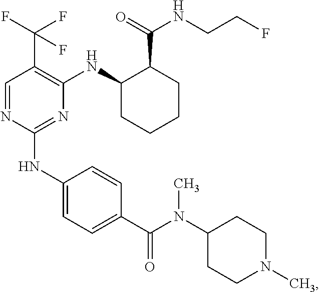 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 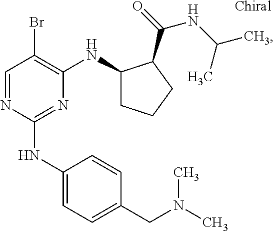 |
| 10 | 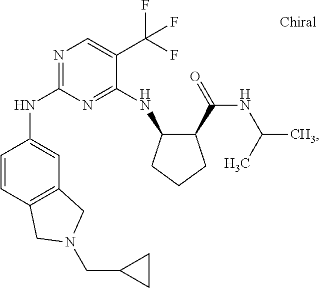 [SMILES: CC(C)NC(=O)[C@H]1CCC[C@H]1Nc1nc(Nc2ccc3c(c2)CN(CC2CC2)C3)ncc1C(F)(F)F ; "Chiral"] |
| 11 | 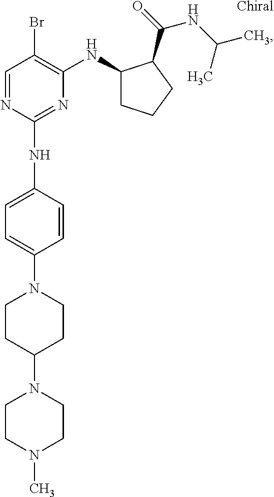 |
| 12 | 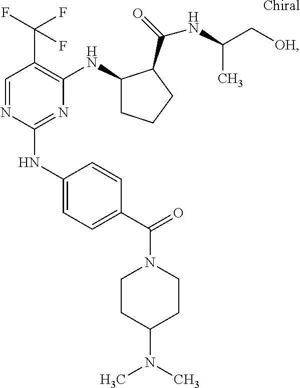 |
| 13 | 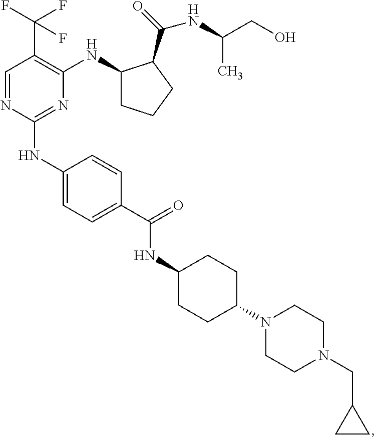 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F ; ","] |
| 14 | 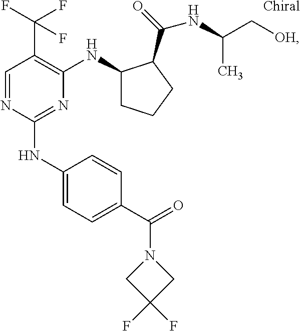 |
| 15 | 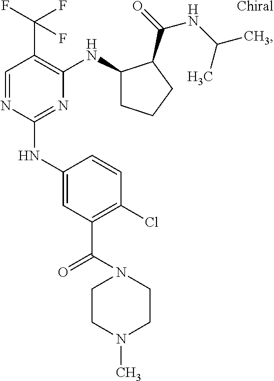 |
| 16 | 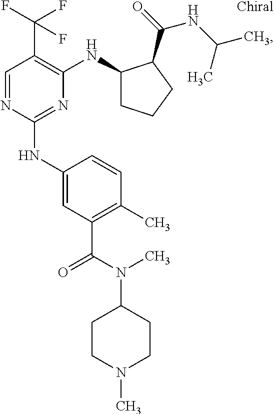 |
| 17 | 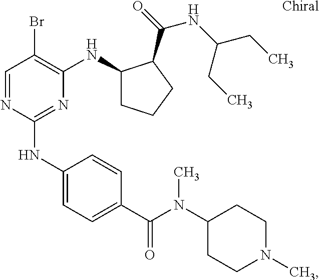 [SMILES: CCC(CC)NC(=O)[C@H]1CCC[C@H]1Nc1nc(Nc2ccc(C(=O)N(C)C3CCN(C)CC3)cc2)ncc1Br ; "Chiral"] |
| 18 | 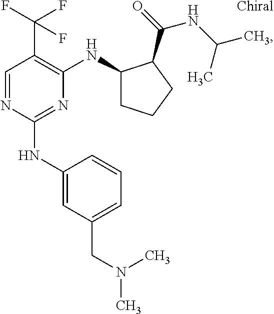 |
| 19 | 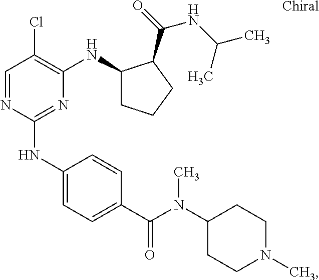 [SMILES: CC(C)NC(=O)[C@H]1CCC[C@H]1Nc1nc(Nc2ccc(C(=O)N(C)C3CCN(C)CC3)cc2)ncc1Cl ; "Chiral"] |
| 20 | 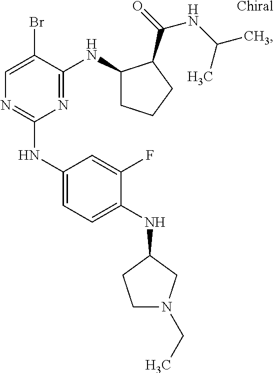 |
| 21 | 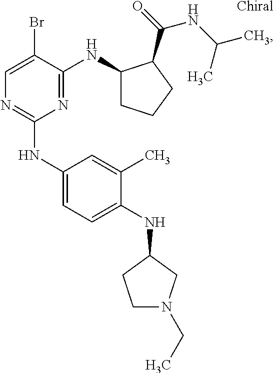 |
| 22 | 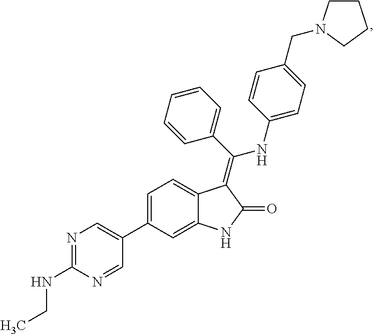 [SMILES: CCNc1ncc(-c2ccc3c(c2)NC(=O)/C3=C(\Nc2ccc(CN3CCCC3)cc2)c2ccccc2)cn1 ; ","] |
| 23 | 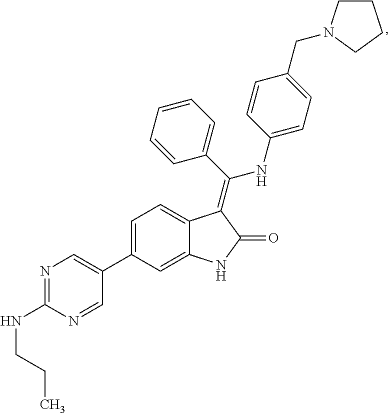 [SMILES: CCCNc1ncc(-c2ccc3c(c2)NC(=O)/C3=C(\Nc2ccc(CN3CCCC3)cc2)c2ccccc2)cn1 ; ","] |
| 24 | 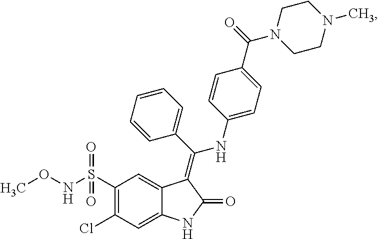 [SMILES: CONS(=O)(=O)c1cc2c(cc1Cl)NC(=O)/C2=C(\Nc1ccc(C(=O)N2CCN(C)CC2)cc1)c1ccccc1] |
| 25 | 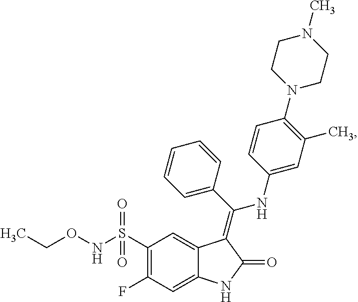 [SMILES: CCONS(=O)(=O)c1cc2c(cc1F)NC(=O)/C2=C(\Nc1ccc(N2CCN(C)CC2)c(C)c1)c1ccccc1] |
| 26 | 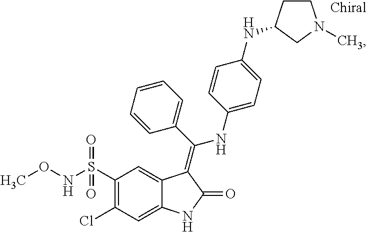 |
| 27 | 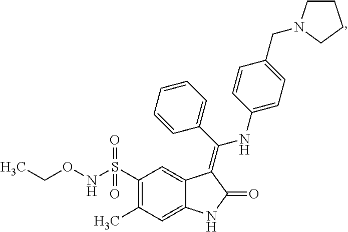 [SMILES: CCONS(=O)(=O)c1cc2c(cc1C)NC(=O)/C2=C(\Nc1ccc(CN2CCCC2)cc1)c1ccccc1 ; ","] |
| 28 | 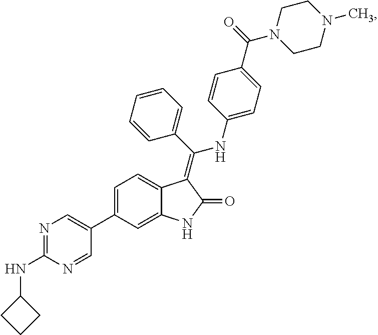 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 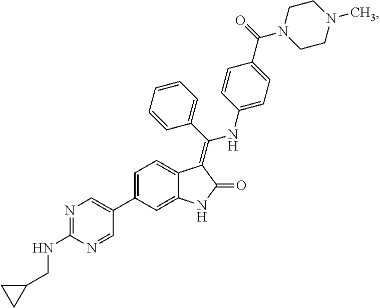 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 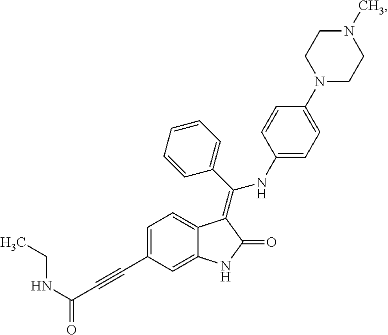 [SMILES: CCNC(=O)C#Cc1ccc2c(c1)NC(=O)/C2=C(\Nc1ccc(N2CCN(C)CC2)cc1)c1ccccc1] |
| 31 | 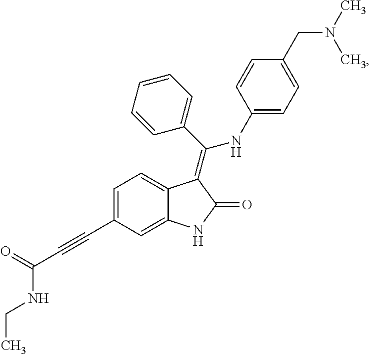 [SMILES: CCNC(=O)C#Cc1ccc2c(c1)NC(=O)/C2=C(\Nc1ccc(CN(C)C)cc1)c1ccccc1] |
| 32 | 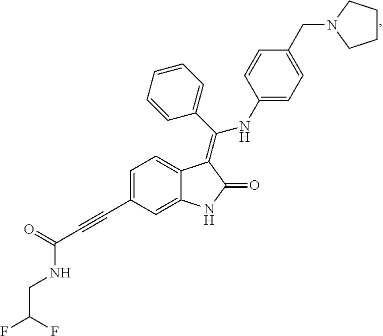 [SMILES: O=C(C#Cc1ccc2c(c1)NC(=O)/C2=C(\Nc1ccc(CN2CCCC2)cc1)c1ccccc1)NCC(F)F ; ","] |
| 33 | 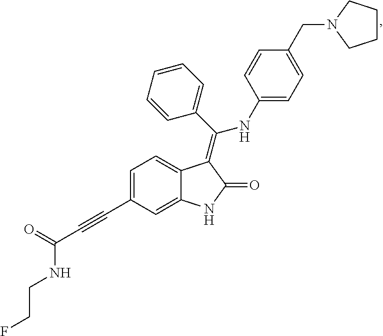 [SMILES: O=C(C#Cc1ccc2c(c1)NC(=O)/C2=C(\Nc1ccc(CN2CCCC2)cc1)c1ccccc1)NCCF ; ","] |
| 34 | 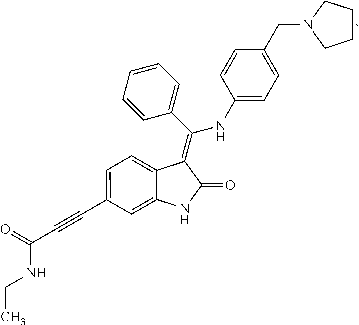 [SMILES: CCNC(=O)C#Cc1ccc2c(c1)NC(=O)/C2=C(\Nc1ccc(CN2CCCC2)cc1)c1ccccc1 ; ","] |
| 35 | 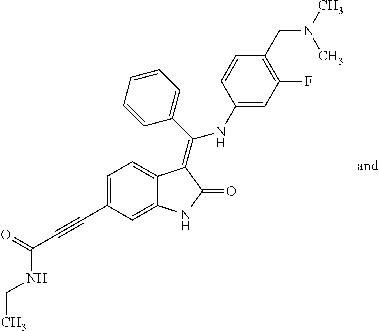 [SMILES: CCNC(=O)C#Cc1ccc2c(c1)NC(=O)/C2=C(\Nc1ccc(CN(C)C)c(F)c1)c1ccccc1 ; "and"] |
| 36 | 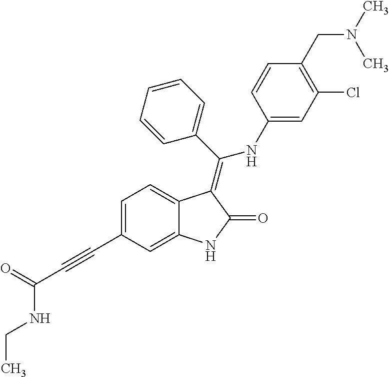 [SMILES: CCNC(=O)C#Cc1ccc2c(c1)NC(=O)/C2=C(\Nc1ccc(CN(C)C)c(Cl)c1)c1ccccc1] |

or a tautomer or pharmaceutically acceptable salt thereof.

19. The method of claim 17 wherein the cell signalling and/or angiogenesis inhibitor is a cell signalling inhibitor which is a compound of formula 3 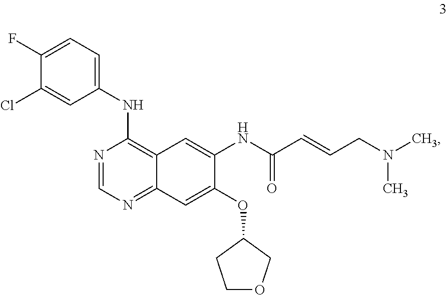 [SMILES: CN(C)C/C=C/C(=O)Nc1cc2c(Nc3ccc(F)c(Cl)c3)ncnc2cc1O[C@H]1CCOC1]
or a tautomer or pharmaceutically acceptable salt thereof, and the Aurora kinase inhibitor is a compound selected from the group consisting of:

| No. | AKI compound |
|---|---|
| 1 | 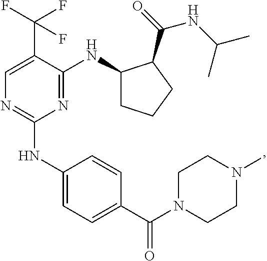 |
| 2 | 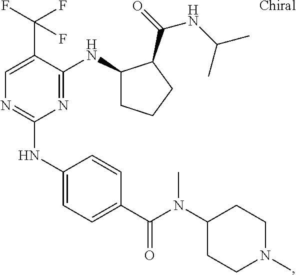 |
| 3 | 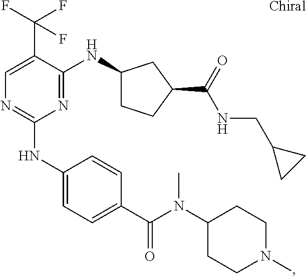 |
| 4 | 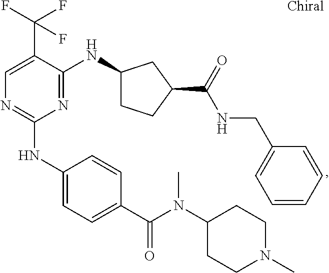 [SMILES: CN1CCC(N(C)C(=O)c2ccc(Nc3ncc(C(F)(F)F)c(N[C@@H]4CC[C@H](C(=O)NCc5ccccc5)C4)n3)cc2)CC1 ; "Chiral" ; ","] |
| 5 | 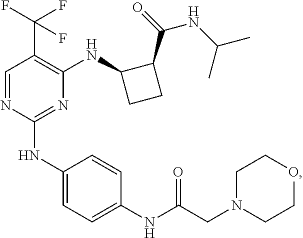 [SMILES: CC(C)NC(=O)[C@H]1CC[C@H]1Nc1nc(Nc2ccc(NC(=O)CN3CCOCC3)cc2)ncc1C(F)(F)F] |
| 6 | 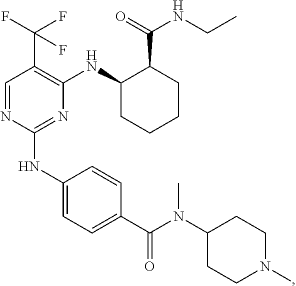 |
| 7 | 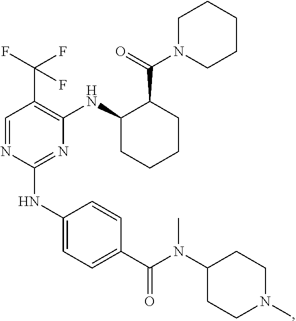 |
| 8 | 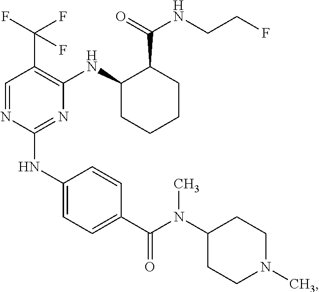 [SMILES: CN1CCC(N(C)C(=O)c2ccc(Nc3ncc(C(F)(F)F)c(N[C@@H]4CCCC[C@@H]4C(=O)NCCF)n3)cc2)CC1] |
| 9 | 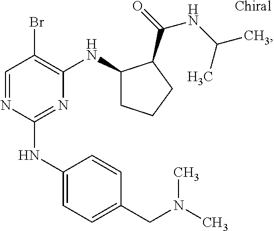 |
| 10 | 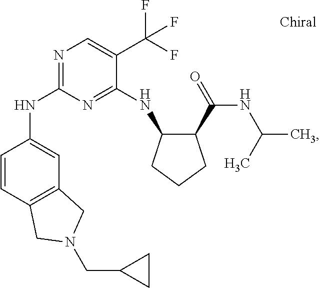 [SMILES: CC(C)NC(=O)[C@H]1CCC[C@H]1Nc1nc(Nc2ccc3c(c2)CN(CC2CC2)C3)ncc1C(F)(F)F ; "Chiral"] |
| 11 | 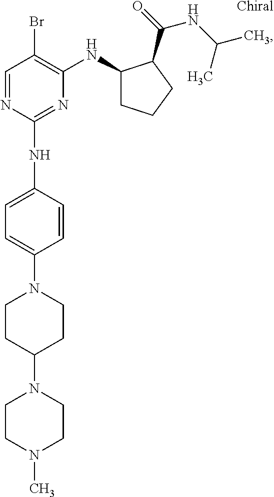 |
| 12 | 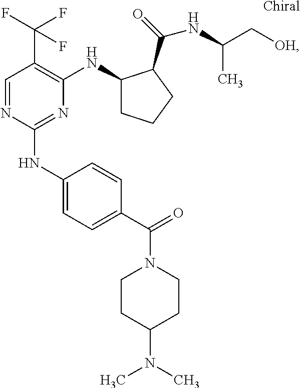 |
| 13 | 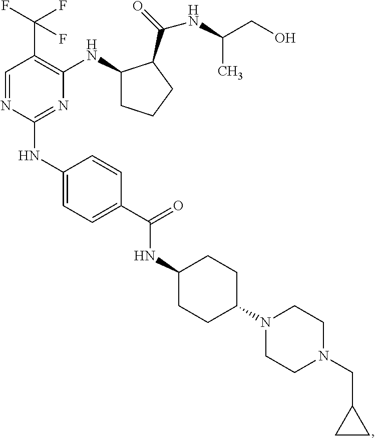 [SMILES: C[C@H](CO)NC(=O)[C@H]1CCC[C@H]1Nc1nc(Nc2ccc(C(=O)N[C@H]3CC[C@H](N4CCN(CC5CC5)CC4)CC3)cc2)ncc1C(F)(F)F ; ","] |
| 14 | 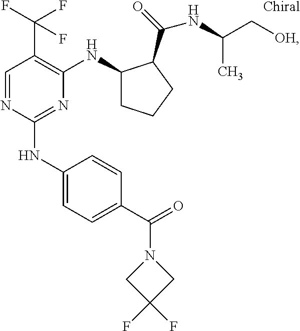 |
| 15 | 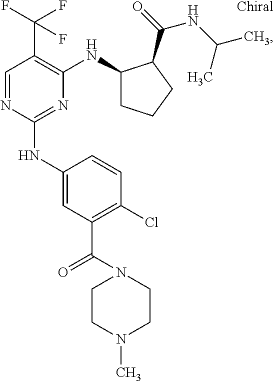 |
| 16 | 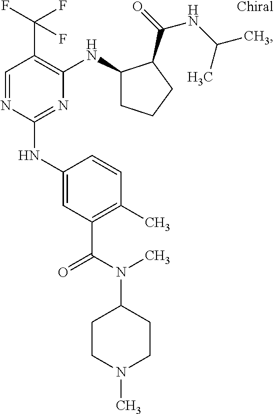 |
| 17 | 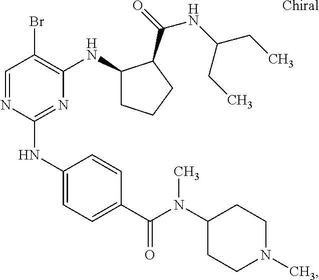 [SMILES: CCC(CC)NC(=O)[C@H]1CCC[C@H]1Nc1nc(Nc2ccc(C(=O)N(C)C3CCN(C)CC3)cc2)ncc1Br ; "Chiral"] |
| 18 | 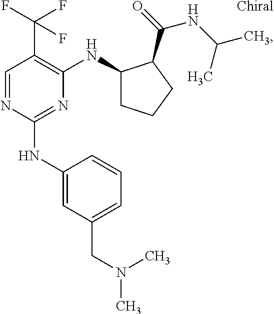 |
| 19 | 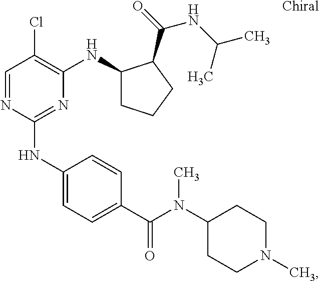 [SMILES: CC(C)NC(=O)[C@H]1CCC[C@H]1Nc1nc(Nc2ccc(C(=O)N(C)C3CCN(C)CC3)cc2)ncc1Cl ; "Chiral"] |
| 20 | 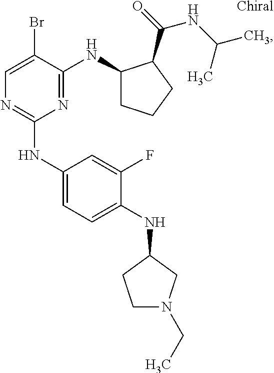 |
| 21 | 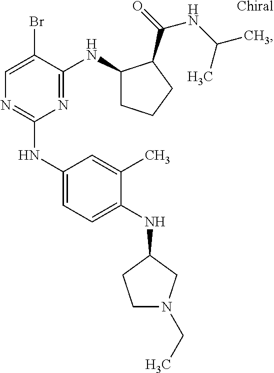 |
| 22 | 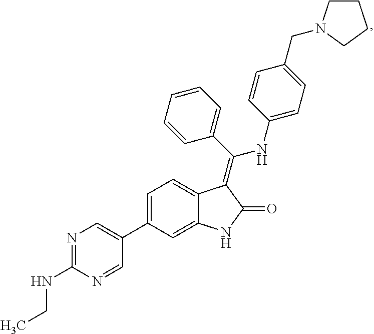 [SMILES: CCNc1ncc(-c2ccc3c(c2)NC(=O)/C3=C(\Nc2ccc(CN3CCCC3)cc2)c2ccccc2)cn1 ; ","] |
| 23 | 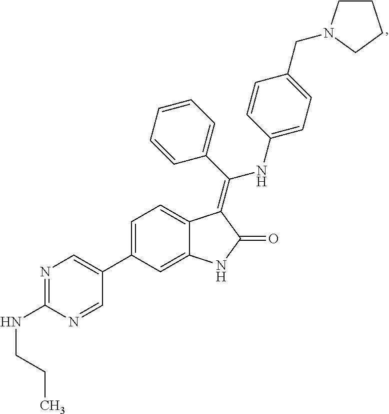 [SMILES: CCCNc1ncc(-c2ccc3c(c2)NC(=O)/C3=C(\Nc2ccc(CN3CCCC3)cc2)c2ccccc2)cn1 ; ","] |
| 24 | 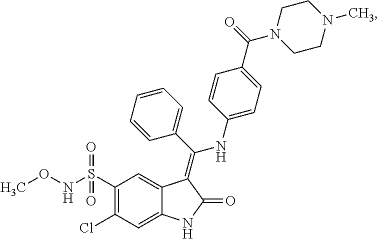 [SMILES: CONS(=O)(=O)c1cc2c(cc1Cl)NC(=O)/C2=C(\Nc1ccc(C(=O)N2CCN(C)CC2)cc1)c1ccccc1] |
| 25 | 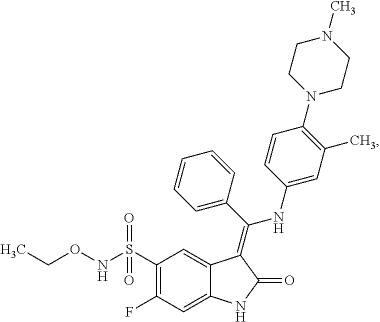 [SMILES: CCONS(=O)(=O)c1cc2c(cc1F)NC(=O)/C2=C(\Nc1ccc(N2CCN(C)CC2)c(C)c1)c1ccccc1] |
| 26 | 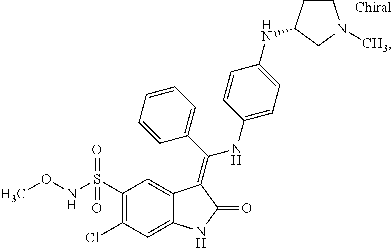 |
| 27 | 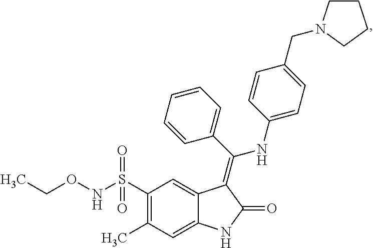 [SMILES: CCONS(=O)(=O)c1cc2c(cc1C)NC(=O)/C2=C(\Nc1ccc(CN2CCCC2)cc1)c1ccccc1 ; ","] |
| 28 | 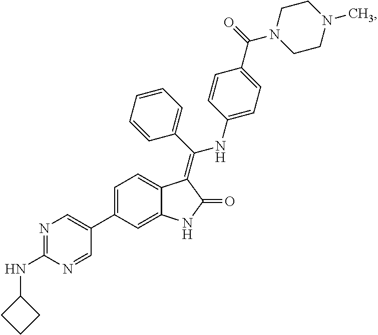 [SMILES: CN1CCN(C(=O)c2ccc(N/C(=C3\C(=O)Nc4cc(-c5cnc(NC6CCC6)nc5)ccc43)c3ccccc3)cc2)CC1] |
| 29 | 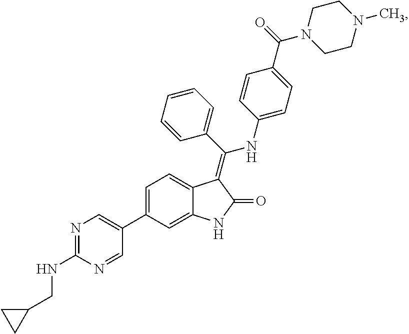 [SMILES: CN1CCN(C(=O)c2ccc(N/C(=C3\C(=O)Nc4cc(-c5cnc(NCC6CC6)nc5)ccc43)c3ccccc3)cc2)CC1] |
| 30 | 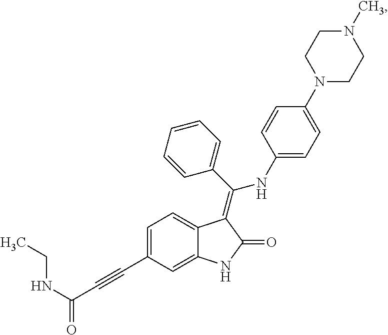 [SMILES: CCNC(=O)C#Cc1ccc2c(c1)NC(=O)/C2=C(\Nc1ccc(N2CCN(C)CC2)cc1)c1ccccc1] |
| 31 | 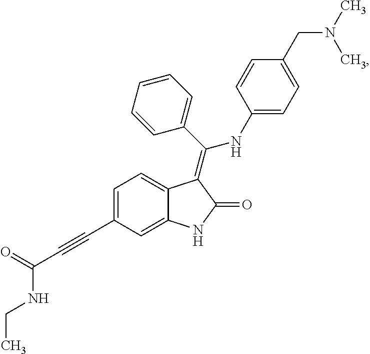 [SMILES: CCNC(=O)C#Cc1ccc2c(c1)NC(=O)/C2=C(\Nc1ccc(CN(C)C)cc1)c1ccccc1] |
| 32 | 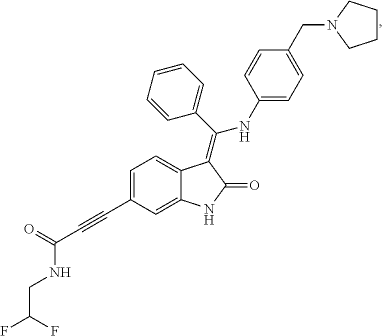 [SMILES: O=C(C#Cc1ccc2c(c1)NC(=O)/C2=C(\Nc1ccc(CN2CCCC2)cc1)c1ccccc1)NCC(F)F ; ","] |
| 33 | 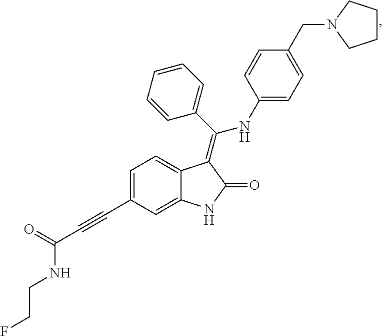 [SMILES: O=C(C#Cc1ccc2c(c1)NC(=O)/C2=C(\Nc1ccc(CN2CCCC2)cc1)c1ccccc1)NCCF ; ","] |
| 34 | 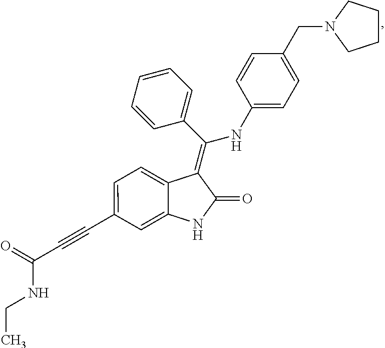 [SMILES: CCNC(=O)C#Cc1ccc2c(c1)NC(=O)/C2=C(\Nc1ccc(CN2CCCC2)cc1)c1ccccc1 ; ","] |
| 35 | 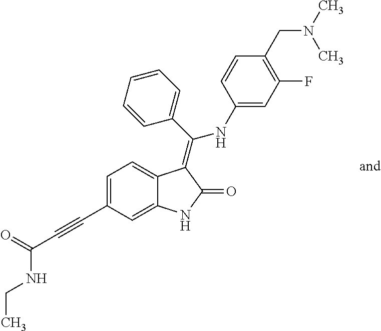 [SMILES: CCNC(=O)C#Cc1ccc2c(c1)NC(=O)/C2=C(\Nc1ccc(CN(C)C)c(F)c1)c1ccccc1 ; "and"] |
| 36 | 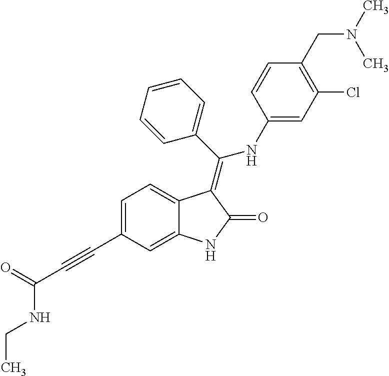 [SMILES: CCNC(=O)C#Cc1ccc2c(c1)NC(=O)/C2=C(\Nc1ccc(CN(C)C)c(Cl)c1)c1ccccc1] |

or a tautomer or pharmaceutically acceptable salt thereof.

20. The method of claim 17, wherein the disease is selected from solid tumours, urogenital cancers, gynecological cancers, lung cancer, gastrointestinal cancers, head and neck cancer, malignant glioblastoma, malignant mesothelioma, non-metastatic or metastatic breast cancer, malignant melanoma or bone and soft tissue sarcomas, and haematologic neoplasias, such as multiple myeloma, acute myelogenous leukemia, chronic myelogenous leukemia, myelodysplastic syndrome and acute lymphoblastic leukemia.
